(19) United States
(12) Patent Application Publication
(10) Pub. No.: US 2025/0221774 A1
(43) Pub. Date: Jul. 10, 2025

(54) IMAGE GUIDED CANCER TREATMENT SYSTEM AND METHODS

(71) Applicant: PROCEPT BioRobotics Corporation, San Jose, CA (US)

(72) Inventors: Peter BENTLEY, Reno, NV (US); James Luis BADIA, Redwood City, CA (US); Qingxiang KE, San Jose, CA (US); Thomas YORKEY, San Jose, CA (US); James DOMINE, Palo Alto, CA (US); Sneha SHIVKUMAR, Saratoga, CA (US)

(73) Assignee: PROCEPT BioRobotics Corporation, San Jose, CA (US)

(21) Appl. No.: 19/089,260

(22) Filed: Mar. 25, 2025

Related U.S. Application Data
(63) Continuation of application No. PCT/US2024/062274, filed on Dec. 30, 2024.
(60) Provisional application No. 63/616,258, filed on Dec. 29, 2023.

(51) Int. Cl. A61B 34/10; A61B 17/3203; G06T 7/73; G06T 15/20; G06T 19/00; G16H 20/40

(52) U.S. Cl. CPC A61B 34/10; G06T 7/73; G06T 15/205; G06T 19/00; G16H 20/40; A61B 17/3203; A61B 2034/105; G06T 2200/24; G06T 2207/20092; G06T 2207/20221; G06T 2207/30096; G06T 2207/30204

(57) ABSTRACT

A diagnostic image to detect cancer is fused with an intraoperative image from an imaging probe such as an ultrasound probe to generate fused image, and the fused image is used to plan a treatment with an energy source. The imaging probe may comprise a transrectal ultrasound (TRUS) probe and the treatment probe may comprise a rotating and translating energy source such as a water jet. The diagnostic and intraoperative images may each comprise three dimensional (3D) images. In some embodiments, the fused image comprise 3D image and two dimensional image slices of the fused 3D image are used for treatment planning. In some embodiments, the patient is treated with an offset configuration, in which the treatment probe and imaging probe are offset relative to each other with respect to a midline of the patient, which can facilitate imaging and provide treatment to different tissue regions.

### RELATED APPLICATIONS

This application is a continuation of International Patent Application No. PCT/US2024/062274, filed Dec. 30, 2024, which claims the benefit under 35 U.S.C. § 119(e) of U.S. Provisional Application No. 63/616,258, filed Dec. 29, 2023, the disclosures of which are incorporated, in their entirety, by this reference.

The subject matter of the present application is related to U.S. Ser. No. 18/163,187, filed on Feb. 1, 2023, entitled “User interface for three dimensional imaging and treatment”, published as US 2024/0252255 on Aug. 1, 2024, and U.S. patent application Ser. No. 18/539,023, filed Dec. 13, 2023, entitled “Ergonomic Surgical Robotic System”, and U.S. patent application Ser. No. 18/539,048, filed Dec. 13, 2023, entitled “User Interface for Surgical Robotic System”, the entire disclosures of which are incorporated herein by reference.

### BACKGROUND

Prior approaches to treating tissue such as cancerous tissue can be less than ideal in at least some respects. Work in relation to the present disclosure suggests that a treatment probe can decrease the visibility of tissue behind a treatment probe as viewed from an imaging probe such as an ultrasound probe. Also, at least some of the prior treatment systems may be less than ideally suited to treat tissue such as anterior tissue of the prostate.

Although the use of fused images such as magnetic resonance imaging (MRI) images and ultrasound images has been proposed for ultrasound biopsies, work in relation to the present disclosure suggests that the prior fused images may be less than ideally suited for treatment with an energy source such as a water jet. Also, because diagnostic images such as MRI images may be taken prior to surgery without probes inserted into the patient, tissue can move once probes have been placed in the patient intraoperatively, which may present challenges in accurately fusing the diagnostic image and the intraoperative image.

### SUMMARY

In some embodiments, a diagnostic image to detect cancer such as an MRI image is fused with an intraoperative image from an imaging probe such as an ultrasound probe to generate a fused image, and the fused image is used to plan a treatment with an energy source. In some embodiments, the intraoperative imaging probe comprises a transrectal ultrasound (TRUS) probe and the treatment probe comprises a rotating and translating energy source such as a water jet.

In some embodiments, an intraoperative three dimensional (3D) image is acquired with a treatment probe and an imaging probe inserted into the patient. While the 3D image can be obtained in many ways, in some embodiments the 3D image is obtained by rotating the probe such as an ultrasound probe to generate a plurality of two dimensional (2D) longitudinal images, and the data from these longitudinal images are combined to generate the 3D intraoperative image. In some embodiments, the 3D diagnostic image data is combined with the 3D intraoperative image data to generate a 3D fused image.

In some embodiments, the shape profile of the treatment probe is known, and information related to the shape profile of the treatment probe is used to fuse the 3D diagnostic image data with the 3D intraoperative image data. In some embodiments, the treatment probe comprises a substantially straight shape profile, and the substantially straight profile of the treatment probe is used to combine the diagnostic image data with the intraoperative image data. While the diagnostic and intraoperative image data can be combined in many ways, in some embodiments a lumen that is curved in the diagnostic image is mapped or otherwise constrained to a straight profile in the fused image, in response to the substantially straight profile of the treatment probe.

In some embodiments, a virtual model of the tissue of an organ such as the prostate is generated from the diagnostic image, such as a 3D diagnostic image. While the virtual model can be configured in many ways, in some embodiments, the virtual model comprises a virtual object such as a mesh of the organ. The deformation of the shape profile of the tissue of the organ in response to the shape profile of treatment probe inserted therein can be modeled to determine the 3D shape profile of the organ with the treatment probe inserted therein. In some embodiments, this deformed shape profile is used to fuse the diagnostic image data with the intraoperative image data.

In some embodiments, a user interface is configured to display a plurality of fused images with a treatment profile overlaid thereon and a plurality of user inputs configured for a user to adjust locations of a plurality of markers overlaid on the plurality of fused images. The plurality of markers may correspond to locations of a treatment profile, locations of tissue structures used to fuse the images. This approach allows the user to adjust the treatment profile and locations of the markers, which can improve image fusion accuracy and the accuracy of the planned treatment.

In some embodiments, an artificial intelligence (AI) algorithm is configured to process one or more of the diagnostic image, the intraoperative image or the fused image, in order to determine the locations of the treatment profiles and markers that are displayed on the plurality of fused images shown on the display. The user interface is configured for the user to adjust the locations of the markers shown on the display, which have been determined with the AI algorithm. In some embodiments, the plurality of fused 2D images shown on the display comprises planes of a 3D fused image, and the treatment profile overlaid on each of the plurality 2D images comprises an intersection of the 3D treatment profile with the corresponding 2D image plane. The plurality of inputs of the user interface allows the user to adjust the locations of the markers overlaid on the 2D fused images that were generated with the AI algorithm.

In some embodiments, the user interface is configured for the user to plan the treatment by viewing a plurality of fused transverse images with the treatment profile and markers overlaid thereon, in which the fused transverse images are obtained from image slices of a 3D fused image. In some embodiments, the user interface is configured for the user to monitor the progress of the treatment with a real time image, such as a real time longitudinal image. In some embodiments, the real time image comprises a real time fused image in which data from the diagnostic image, such as lesion data, is projected onto a real time longitudinal image, such as a real time sagittal TRUS image, to generate the real time fused image.

In some embodiments, the patient is treated with an offset configuration, in which the treatment probe and imaging probe are offset relative to each other with respect to a midline of the patient in order to treat tissue anterior to the treatment probe with the imaging probe located posteriorly relative to the treatment probe. In some embodiments, this offset configuration improves visibility of the tissue located anteriorly relative to the treatment probe and the imaging probe.

In some embodiments, a first region of tissue is treated with the energy source, in which the tissue of the first region has been diagnosed as cancerous, and a second region comprising a tissue that has not been diagnosed as cancerous is treated after the first region of tissue. In some embodiments, this sequential approach can decrease movement of tissue of the first region as compared to treating both regions simultaneously, which can improve the accuracy of treating the first region. In some embodiments, a third region of tissue is treated with the energy source, in which the tissue of the third region has been diagnosed as cancerous, and a fourth region comprising a tissue that has not been diagnosed as cancerous is treated after the third region of tissue. In some embodiments, this sequential approach can decrease movement of tissue of the third region as compared to treating both the third and fourth regions simultaneously, which can improve the accuracy of treating the third region.

In some embodiments, one or more of the treatment probe or the imaging probe is configured to move relative to the other probe while both probes are inserted into the patient and tissue is treated on a first side of the patient with a first configuration of the treatment probe and the imaging probe and the tissue is treated on a second side of the patient with a second configuration of the treatment probe and the imaging probe. In some embodiments, the treatment probe is located on a first side of the patient to treat tissue on a second side of the patient in the first configuration and on the second side of the patient to treat tissue on the first side of the patient in the second configuration. In some embodiments, the first side of the patient is on a first side of a midline of the patient and the second side of the patient is located on a second side of the midline of the patient.

### INCORPORATION BY REFERENCE

All patents, applications, and publications referred to and identified herein are hereby incorporated by reference in their entirety, and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a front view of a system for performing tissue resection in a patient, in accordance with some embodiments;

FIG. 2 schematically illustrates a system for performing tissue resection in a patient, in accordance with some embodiments;

FIG. 3A shows a superior view of an arrangement of probes, in accordance with some embodiments;

FIG. 3B shows a longitudinal view such as a sagittal view of an arrangement of probes, in accordance with some embodiments;

FIG. 3C shows a perspective view of an arrangement of probes, in accordance with some embodiments;

FIG. 3D shows a treatment probe axis and an imaging probe axis skewed with respect to each other by an angle, such that the treatment probe and the imaging probe do not extend along a common plane, in accordance with some embodiments;

FIG. 4A shows a superior view of transverse image planes with respect to an axis of the treatment probe, in accordance with some embodiments;

FIG. 4B shows a three-dimensional view of transverse image planes with respect to a treatment probe, in accordance with some embodiments;

FIG. 4C shows a longitudinal view such as a sagittal view of a treatment probe, in accordance with some embodiments;

FIG. 4D shows transverse image planes rotated with respect to an elongate axis of a probe, in accordance with some embodiments;

FIG. 5 shows treatment probe and corresponding movements of an energy source, in accordance with some embodiments;

FIGS. 6A to 6C show a user interface and transverse images that show movement of a probe location in the transverse images, in accordance with some embodiments;

FIG. 7A shows a user interface with a three dimensional view for three dimensional treatment planning, in accordance with some embodiments;

FIG. 7B shows a longitudinal image such as a sagittal image for three dimensional treatment planning, in accordance with some embodiments;

FIGS. 7C and 7D show a plurality of rotational angles relative to the treatment probe that can be used to generate a plurality of longitudinal images, in accordance with some embodiments;

FIG. 8A shows a diagnostic image to detect cancer, in accordance with some embodiments;

FIG. 8B shows an intra operative image such as an ultrasound image, in accordance with some embodiments;

FIG. 8C shows a fused image, in accordance with some embodiments;

FIG. 9A shows a fused image and a first treatment plan to treat a lesion, in accordance with some embodiments;

FIG. 9B shows a treatment plan to treat a second region of tissue and a second treatment plan to treat a second rejection of tissue after the first region has been treated and one or more probes repositioned, in accordance with some embodiments;

FIG. 9C shows tissue after completion of the first treatment and the second treatment, in accordance with some embodiments;

FIG. 10A shows a first configuration of a treatment probe and an imaging probe to treat tissue on a first side of the patient, in accordance with some embodiments;

FIG. 10B shows a first sweep angle and first targetable areas with the probe in the first configuration, in accordance with some embodiments;

FIG. 10C shows a second configuration of the treatment probe and the imaging probe of FIG. 10A to treat tissue on a second side of the patient, in accordance with some embodiments;

FIG. 10D shows a sweep angle and targetable areas with a second sweep angle of the probe in the second configuration, in accordance with some embodiments;

FIG. 11 shows an arrangement of a treatment probe and an imaging probe with an offset between the treatment probe and the imaging probe, in accordance with some embodiments;

FIG. 12A shows a targeted treatment area, in accordance with some embodiments;

FIG. 12B shows tissue treatment regions with the first configuration of the probes and the second configuration of the probes shown in FIGS. 10A-10D, in accordance with some embodiments;

FIG. 13 shows a method of training an artificial intelligence (AI) algorithm, in accordance with some embodiments;

FIG. 14 shows a two-dimensional convolutional neural network, in accordance with some embodiments;

FIG. 15A shows rotation of an imaging probe about a longitudinal axis to acquire a plurality of longitudinal images, in accordance with some embodiments;

FIG. 15B shows a longitudinal image among the plurality of longitudinal images of FIG. 15A, in accordance with some embodiments;

FIG. 16A shows a treatment plan and user interface along a longitudinal image such as a sagittal image, in accordance with some embodiments;

FIG. 16B shows a treatment plan and user interface along a transverse image, in accordance with some embodiments;

FIG. 17A shows projection of a lesion from a first longitudinal image to a second longitudinal image using a treatment probe as a reference, in accordance with some embodiments;

FIG. 17B shows an axial view of three planes as in FIG. 17A viewed from a corresponding transverse image, in accordance with some embodiments;

FIG. 17C shows radii and corresponding angles and the calculation of a projection of the lesion from the first longitudinal image to the second longitudinal image, in accordance with the embodiments of FIGS. 17A and 17B;

FIG. 18A shows two transverse images and projection of a lesion from a first image to a second image, in accordance with some embodiments;

FIG. 18B shows locations of the transverse images of FIG. 18A on a longitudinal image such as a sagittal image, in accordance with some embodiments;

FIG. 19A shows a lesion, e.g. a ghost lesion, projected onto a longitudinal image of a treatment planning user interface, in accordance with some embodiments;

FIG. 19B shows a lesion, e.g. a ghost lesion, projected onto a transverse image of a treatment planning user interface, in accordance with some embodiments;

FIG. 20A shows a prostate and associated tissue structures, in accordance with some embodiments;

FIG. 20B shows a urethra of prostate prior to insertion of a probe;

FIG. 20C shows a urethra of a prostate with a probe inserted therein; and

FIG. 21 shows a method of generating a treatment plan, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein. Although reference is made to cancer treatment, the presently disclosed methods and apparatus are well suited for treating other types of tissue, such as benign tissue as described herein.

The presently disclosed systems and methods are well suited for use with many probes and diagnostic and surgical procedures. Although reference is made to a treatment probe comprising an energy source for prostate surgery and a transrectal ultrasound (“TRUS”) probe, the present disclosure is well suited for use with many types of probes inserted into many types of tissues, organs, cavities and lumens, such as brain, heart, lung, intestinal, eye, skin, kidney, liver, pancreas, stomach, uterus, ovaries, testicles, bladder, ear, nose, mouth, tumors, cancers, soft tissues such as bone marrow, adipose tissue, muscle, glandular and mucosal tissue, spinal and nerve tissue, cartilage, hard biological tissues such as teeth, bone, and lumens such as vascular lumens, nasal lumens and cavities, sinuses, colon, urethral lumens, gastric lumens, airways, esophageal lumens, trans esophageal, intestinal lumens, anal lumens, vaginal lumens, trans abdominal, abdominal cavities, throat, airways, lung passages, and surgery such as kidney surgery, ureter surgery, kidney stones, prostate surgery, tumor surgery, cancer surgery, brain surgery, heart surgery, eye surgery, conjunctival surgery, liver surgery, gall bladder surgery, bladder surgery, spinal surgery, orthopedic surgery, arthroscopic surgery, liposuction, colonoscopy, intubation, minimally invasive incisions, minimally invasive surgery, and others.

The presently disclosed systems and methods are well suited for combination with prior probes such as imaging probes and treatment probes. Examples of such probes include laser treatment probes, water jet probes, RF treatment probes, radiation therapy probes, ultrasound treatment probes, phaco-emulsification probes, imaging probes, endoscopic probes, resectoscope probes, ultrasound imaging probes, A-scan ultrasound probes, B-scan ultrasound probes, 3D ultrasound probes, Doppler ultrasound probes, transrectal ultrasound probes, transvaginal ultrasound probes, longitudinal plane ultrasound imaging probes, sagittal plane ultrasound imaging probes, transverse plane ultrasound imaging probes, and transverse and longitudinal plane (e.g. sagittal plane) ultrasound imaging probes, for example.

The presently disclosed systems, methods and apparatuses are well suited for combination with many prior surgical procedures, such as water jet enucleation of the prostate, transurethral resection of the prostate (TURP), holmium laser enucleation of the prostate (HOLEP), prostate brachytherapy and with surgical robotics systems and automated surgical procedures. The following patent applications describe examples of systems, methods, probes and procedures suitable for incorporation in accordance with the present disclosure: PCT/US2013/028441, filed Feb. 28, 2013, entitled “AUTOMATED IMAGE-GUIDED TISSUE RESECTION AND TREATMENT, published as WO 2013/130895; PCT/US2014/054412, filed Sep. 5, 2014, entitled “AUTOMATED IMAGE-GUIDED TISSUE RESECTION AND TREATMENT”, published as WO 2015/035249; PCT/US2015/048695, filed Sep. 5, 2015, entitled “PHYSICIAN CONTROLLED TISSUE RESECTION INTEGRATED WITH TREATMENT MAPPING OF TARGET ORGAN IMAGES”, published as WO2016037137; PCT/US2019/038574, filed Jun. 21, 2019, entitled “ARTIFICIAL INTELLIGENCE FOR ROBOTIC SURGERY”, published as WO2019246580A1 on Dec. 26, 2019; PCT/US2020/021756, filed Mar. 9, 2020, entitled “ROBOTIC ARMS AND METHODS FOR TISSUE RESECTION AND IMAGING”, published as WO/2020/181290; PCT/US2020/058884, filed on Nov. 4, 2020, entitled “SURGICAL PROBES FOR TISSUE RESECTION WITH ROBOTIC ARMS”, published as WO/2021/096741; PCT/US2021/070760, filed on Jun. 23, 2021, entitled “INTEGRATION OF ROBOTIC ARMS WITH SURGICAL PROBES”, published as WO/2021/263276; PCT/US2021/038175, filed on Jun. 21, 2021, entitled “SYSTEMS AND METHODS FOR DEFINING AND MODIFYING RANGE OF MOTION OF PROBE USED IN PATIENT TREATMENT”, published as WO/2021/262565; and PCT/US2022/025617, filed on Apr. 20, 2022, entitled “SURGICAL PROBE WITH INDEPENDENT ENERGY SOURCES”, published as WO/2022/226103 on Oct. 27, 2022; the entire disclosures of which are incorporated herein by reference.

In some embodiments, improved positional accuracy is provided for placement of an energy source and imaging probe. The energy source may comprise any suitable energy source, such as an electrode, a radiofrequency (RF) electrode, a loop electrode, an RF loop electrode, laser source, mechanical sheer, a mechanical energy source, a radiation energy source, a thermal energy source, a vibrational energy source, an ultrasound probe, cavitating ultrasound probe, a water jet, a variable pressure water jet, a pressure controlled water jet, a flow rate controlled water jet, a fluctuating pressure water jet, a fixed pressure water jet, plasma, steam, a morcellator, a trans urethral needle, photo ablation, water jet evacuation. The energy source can be combined with other treatments and compounds, such as compounds for treatment, hemostasis, photochemical treatment, or contrast agents for imaging. The imaging probe may comprise any suitable probe, such as endoscopic probes, resectoscope probes, ultrasound imaging probes, A-scan ultrasound probes, B-scan ultrasound probes, Doppler ultrasound probes, transrectal ultrasound probes, transvaginal ultrasound probes, longitudinal plane (e.g. sagittal plane) ultrasound imaging probes, transverse plane ultrasound imaging probes, and transverse and longitudinal (e.g. sagittal) plane ultrasound imaging probes, for example.

The probes comprising the energy source and the imaging probes can be configured in many ways, and each may comprise one or more fiducials for determining a position and orientation of a respective probe.

Although the present disclosure refers to treatment planning with a probe inserted into the patient, the presently disclosed systems and methods are well suited for pre-treatment planning. In some embodiments, the treatment planning is performed without a probe inserted into the patient, for example with images obtained prior to one or more of the treatment probe or the image probe being inserted into the patient.

Although the present disclosure refers to an imaging probe that is separated from the treatment probe, the presently disclosed methods and apparatus are well suited for use with a treatment probe that comprises an imaging probe. In some embodiments the imaging probe is located on the treatment probe, for example. Examples of an imaging device such as an imaging array located on a rotating and translating treatment probe are described in PCT/US2013/028441, filed Feb. 28, 2013, entitled “AUTOMATED IMAGE-GUIDED TISSUE RESECTION AND TREATMENT, published as WO 2013/130895, the entire disclosure of which has been incorporated herein by reference.

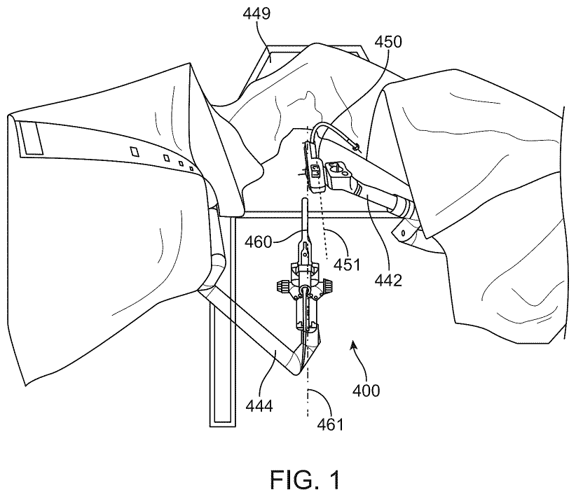

FIG. 1 shows an exemplary embodiment of a system 400 for performing treatment of a patient. The system 400 may comprise a treatment probe 450 as described herein and an imaging probe 460 as described herein. The treatment probe 450 may be coupled to a first arm 442, and the imaging probe 460 coupled to a second am 444. One or both of the first arm 442 and the second arm 444 may comprise robotic arms whose movements may be controlled by one or more computing devices operably coupled with the arms. The treatment probe 450 may comprise a device for removing target tissue from a target site within a patient. The treatment probe 450 may be configured to deliver energy from the treatment probe 450 to the target tissue sufficient for removing the target tissue, and the energy from the energy source may comprise any suitable energy as described herein. For example, the treatment probe 450 may comprise an electrosurgical ablation device, a laser ablation device, a transurethral needle ablation device, a water jet ablation device, a steam ablation device, a high-intensity focused ultrasound (HIFU) device, or any combination thereof. The imaging probe 460 may be configured to deliver energy from the imaging probe 460 to the target tissue sufficient for imaging the target tissue. The imaging probe 460 may comprise an ultrasound probe, a magnetic resonance probe, an endoscope, or a fluoroscopy probe, for example. The first arm 442 and the second arm 444 may be configured to be independently adjustable, adjustable according to a fixed relationship, adjustable according to a user selected relationship, independently lockable, or simultaneously lockable, or any combination thereof. The first arm 442 and the second arm 444 may have multiple degrees of freedom, for example six degrees of freedom, to manipulate the treatment probe 450 and the imaging probe 460, respectively. The treatment system 400 may be used to perform tissue resection in an organ of a patient, such a prostate of a patient. The patient may be positioned on a patient support 449 such as a bed, a table, a chair, or a platform. The treatment probe 450 may be inserted into the target site of the patient along an axis of entry that coincides with the elongate axis 451 of the treatment probe. For example, the treatment probe 450 may be configured for insertion into the urethra of the patient, so as to position an energy delivery region of the treatment probe within the prostate of the patient. The imaging probe 460 may be inserted into the patient at the target site or at a site adjacent the target site of the patient, along an axis of entry that coincides with the elongate axis 461 of the imaging probe. For example, the imaging probe 460 may comprise a transrectal ultrasound (TRUS) probe, configured for insertion into the rectum of the patient to view the patient's prostate and the surrounding tissues. As shown in FIG. 1, the first arm 442 and the second arm 444 may be covered in sterile drapes to provide a sterile operating environment, keep the robotic arms clean, and reduce risks of damaging the robotic arms. Further details regarding the various components of the system 400 suitable for incorporation with embodiments as disclosed herein may be found in U.S. Pat. Nos. 7,882,841, 8,814,921, 9,364,251, and PCT Publication No. WO2013/130895, the entire disclosures of which are incorporated herein by reference.

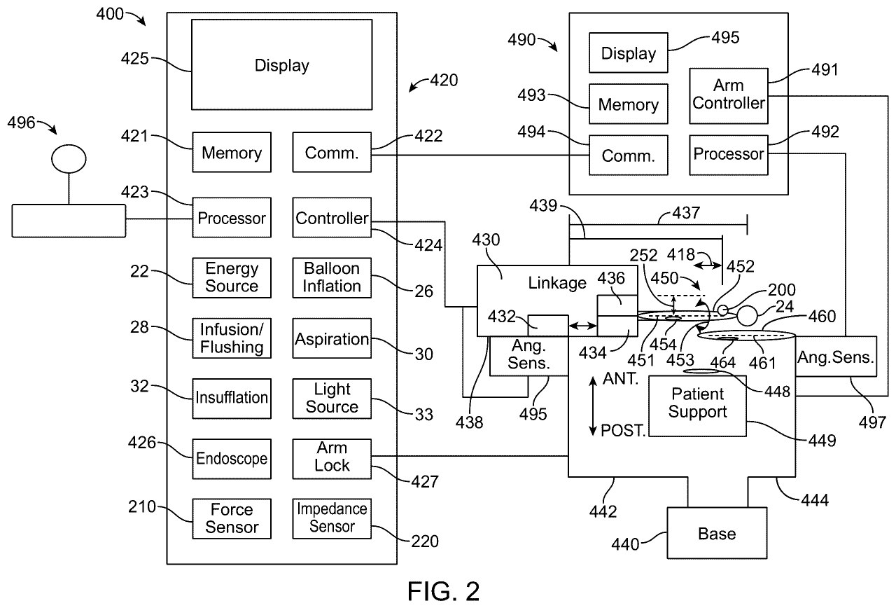

FIG. 2 schematically illustrates embodiments of the system 400 for treating tissue in a patient with an energy source, for example for performing tissue resection. The system 400 may comprise a treatment probe 450 as described herein and may optionally comprise an imaging probe 460. The treatment probe 450 is coupled to a console 420 and a linkage 430. The linkage 430 may comprise one or more components of the robotic arm 442. The imaging probe 460 is coupled to an imaging console 490. The imaging probe may be coupled to the second robotic arm 444, for example. The patient treatment probe 450 and the imaging probe 460 can be coupled to a common base 440. The patient is supported with the patient support 449. The treatment probe 450 is coupled to the base 440 with a first arm 442. The imaging probe 460 is coupled to the base 440 with a second arm 444. One or both of the first arm 442 and the second arm 444 may comprise robotic arms whose movements may be controlled by one or more computing devices operably coupled with the arms, as described in further detail herein.

Although reference is made to a common base, the robotic arms can be coupled to a bed rail, a console, or any suitable supporting structure to support the base of the robotic arm.

In some embodiments, the system 400 comprises electromagnetic (“EM”) sensors, such as coils and magnets to determine the position of one or more of the treatment probe 450 or the imaging probe 460. The EM sensor may comprise one or more of a coil, a magnet, or an electromagnet, for example. In some embodiments, the patient support 449 comprises an EM sensor 448 configured to determine one or more of a position or an orientation of the treatment probe 450 or the imaging probe 460. In some embodiments, the treatment probe 450 comprises an EM sensor 454 configured to determine one or more of a position or an orientation of the treatment probe 450. In some embodiments, the imaging probe 460 comprises an EM sensor 464 configured to determine one or more of a position or an orientation of the imaging probe.

While the EM sensor 449 can be configured in many ways, in some embodiments, the EM sensor 449 is configured to electromagnetically couple to one or more of the sensor 454 or the sensor 464. In some embodiments, the sensor 454 comprises a coil and the sensor 464 comprises a coil and the EM sensor 449 is configured to determine the one or more of the position or the orientation of the treatment probe 450 in response to a magnetic field received at sensor 449. In some embodiments, one or more of sensor 448, sensor 454 or sensor 464 are coupled to circuitry configured to determine the one or more of the position or the orientation of the one or more of treatment probe 450 or the imaging probe 460 in response to magnetic signals received at sensor 448. In some embodiments, sensors 448, 454, 464 are coupled to a processor such as processor 423 to selectively control current through a coil of sensor 454 and a coil of sensor 464 in order to determine the one or more of the position or the orientation of the probe 450 and probe 460 while both probes 450, 460, and both sensors 454, 464 have been inserted into the patient. Examples of commercially available EM sensors suitable for incorporation with the present disclosure include sensors and components from BK Medical, a part of GE Healthcare, Lucent Medical Systems, and the UroNav navigation sensor from Philips, for example.

In some embodiments, system 400 comprises a user input device 496 coupled to processor 423 for a user to manipulate the surgical instrument on the robotic arm. A user input device 496 can be located in any suitable place, for example, on a console, on a robotic arm, on a mobile base, and there may be one, two, three, four, or more user input devices used in conjunction with the system 400 to either provide redundant avenues of input, unique input commands, or a combination. In some embodiments, the user input device comprises a controller to move the end of the treatment probe or the imaging probe with movements in response to mechanical movements of the user input device. The end of the probe can be shown on the display 425 and the user can manipulate the end of the probe. For example, the user input device may comprise a 6 degree of freedom input controller in which the user is able to move the input device with 6 degrees of freedom, and the distal end of the probe moves in response to movements of the controller. In some embodiments, the 6 degrees of freedom comprise three translational degrees of freedom and three rotational degrees of freedom. Although reference is made to a user input device with 6 degrees of freedom, any suitable user input device can be used such as a pointing device or a touch screen display, for example. The processor can be configured with instructions for the probe control to switch between automated image guidance treatment with the energy source and treatment with the energy source with user movement of the user input device, for example.

While the user input device can be configured in many ways, in some embodiments the user input device is configured to allow the user to rotate and translate the energy source on the distal end of the probe by rotating and translating the carrier that carries the energy source while other components of the treatment probe remain fixed. Alternatively or in combination, the user input device 496 can be configured to allow the user to control placement of the probes within the patient, by moving a proximal end of the probe, for example by moving a proximal end of the probe. In some embodiments, arm 442 or arm 444 comprises a robotic arm as described herein, and the user input device 496 is configured to receive inputs to move the proximal end of the treatment probe 450 or the imaging probe 460. In some embodiments, the user input device 496 is configured to receive a user input to select which probe to move in response to user inputs and to receive user inputs controlling the position of arm 442 or arm 444. In some embodiments, one or more of arm 442 or arm 444 is configured to position the proximal end of the respective probe, e.g. probe 450 or probe 460 with 6 degrees of freedom, such as 3 translational degrees of freedom and 3 rotational degrees of freedom.

The patient is placed on the patient support 449, such that the treatment probe 450 and ultrasound probe 460 can be inserted into the patient. The patient can be placed in one or more of many positions such as prone, supine, upright, or inclined, for example. In some embodiments, the patient is placed in a lithotomy position, and stirrups may be used, for example as shown in FIG. 1. In some embodiments, the treatment probe 450 is inserted into the patient in a first direction on a first side of the patient, and the imaging probe is inserted into the patient in a second direction on a second side of the patient. For example, the treatment probe can be inserted from an anterior side of the patient into a urethra of the patient, and the imaging probe can be inserted trans-rectally from a posterior side of the patient into the intestine of the patient. Because tissue associate with the urethra is soft, the urethral tissue can be manipulated to allow the probes to be inserted into the patient in a similar direction. The treatment probe and imaging probe can be placed in the patient with one or more of urethral tissue, urethral wall tissue, prostate tissue, intestinal tissue, or intestinal wall tissue extending therebetween.

Although FIG. 2 shows the treatment probe 450 and the imaging probe 460 facing towards each other, in some embodiments the treatment probe and the imaging probe face in the same direction in a substantially parallel configuration as described herein.

The treatment probe 450 and the imaging probe 460 can be inserted into the patient in one or more of many ways. In some embodiments, the treatment probe 450 comprises a handpiece and an elongate shaft sized for insertion into the patient, and the handpiece is inserted into the patient prior to coupling the handpiece to arm 442. While the treatment probe and the imaging probe can be inserted in any order, in some embodiments the imaging probe 460 is inserted into the patient prior to inserting the treatment probe 460.

During insertion, each of the first and second arms may comprise a substantially unlocked configuration such the treatment or imaging probe can be desirably rotated and translated in order to insert the probe into the patient. When the probe has been inserted to a desired location, the arm can be locked. In the locked configuration, the probes can be oriented in relation to each other in one or more of many ways, such as parallel, skew, horizontal, oblique, or non-parallel, for example. It can be helpful to determine the orientation of the probes with angle sensors as described herein, in order to map the image date of the imaging probe to treatment probe coordinate references. Having the tissue image data mapped to treatment probe coordinate reference space can allow accurate targeting and treatment of tissue identified for treatment by an operator such as the physician.

In some embodiments, the treatment probe 450 is coupled to the imaging probe 460 in order to align the treatment with probe 450 based on images from imaging probe 460. The coupling can be achieved with the common base 440 as shown. Alternatively or in combination, the treatment probe and/or the imaging probe may comprise magnets to hold the probes in alignment through tissue of the patient. In some embodiments, the first arm 442 is a movable and lockable arm such that the treatment probe 450 can be positioned in a desired location in a patient. When the probe 450 has been positioned in the desired location of the patient, the first arm 442 can be locked with an arm lock 427. The imaging probe can be coupled to base 440 with the second arm 444, which can be used to adjust the alignment of the imaging probe when the treatment probe is locked in position. The second arm 444 may comprise a lockable and movable arm under control of the imaging system or of the console and of the user interface, for example. The movable arm 444 may be micro-actuatable so that the imaging probe 460 can be adjusted with small movements, for example a millimeter or so in relation to the treatment probe 450.

In some embodiments, the treatment probe 450 and the imaging probe 460 are coupled to angle sensors so that the treatment can be controlled based on the alignment of the imaging probe 460 and the treatment probe 450. A first angle sensor 495 may be coupled to the treatment probe 450 with a support 438. A second angle sensor 497 may be coupled to the imaging probe 460. The angle sensors may comprise one or more of many types of angle sensors. For example, the angle sensors may comprise goniometers, accelerometers and combinations thereof. In some embodiments, the first angle sensor 495 comprises a 3-dimensional accelerometer to determine an orientation of the treatment probe 450 in three dimensions. In some embodiments, the second angle sensor 497 comprises a 3-dimensional accelerometer to determine an orientation of the imaging probe 460 in three dimensions. Alternatively or in combination, the first angle sensor 495 may comprise a goniometer to determine an angle of treatment probe 450 along an elongate axis 451 of the treatment probe. The second angle sensor 497 may comprise a goniometer to determine an angle of the imaging probe 460 along an elongate axis 461 of the imaging probe 460. The first angle sensor 495 is coupled to a controller 424 of the treatment console 420. The second angle sensor 497 of the imaging probe is coupled to a processor 492 of the imaging console 490. Alternatively or in combination, the second angle sensor 497 may be coupled to the controller 424 of the treatment console 420.

The console 420 comprises a display 425 coupled to a processor system in components that are used to control treatment probe 450. The console 420 comprises a processor 423 having a memory 421. Communication circuitry 422 is coupled to processor 423 and controller 424. Communication circuitry 422 is coupled to the imaging console 490 via the communication circuitry 494 of the imaging console. Arm lock 427 of console 420 may be coupled to the first arm 442 to lock the first arm or to allow the first arm to be freely movable to insert probe 450 into the patient.

Optionally, the console 420 may comprise components of an endoscope 426 that is coupled to anchor 24 of the treatment probe 450. Endoscope 426 can comprise components of console 420 and an endoscope insertable with treatment probe 450 to treat the patient.

In some embodiments, the console 420 comprises impedance sensor circuitry 220 coupled to the energy source to measure impedance of tissue treated with energy from the energy source. In some embodiments, the energy source comprises an electrode and the electrode comprises an impedance sensor. In some embodiments, the processor is configured with instructions to adjust an amount of energy from the energy source in response to an amount of impedance. In some embodiments, the processor is configured with instructions to adjust an amount of deflection of the extension and offset of the energy source from the elongate axis in response to impedance.

In some embodiments, the console 420 comprises force sensor circuitry 210 coupled to a force sensor on the treatment probe. The force sensor can be coupled to the extension to measure tissue resistance related to deflection of the extension, for example. In some embodiments, the force sensor is coupled to the link to measure tissue resistance related to movement of the energy source away from the elongate axis. In some embodiments, the force sensor is coupled to the energy source to measure tissue resistance related to a positioning distance of the energy source from the elongate axis. In some embodiments, the force sensor is configured to measure tissue resistance related to an amount of energy delivery from the energy source.

Optionally, the console 420 may comprise one or more of modules operably coupled with the treatment probe 450 to control an aspect of the treatment with the treatment probe. For example, the console 420 may comprise one or more of an energy source 22 to provide energy to the treatment probe, balloon inflation control 26 to affect inflation of a balloon used to anchor the treatment probe at a target treatment site, infusion/flushing control 28 to control infusion and flushing of the probe, aspiration control 30 to control aspiration by the probe, insufflation control 32 to control insufflation of the target treatment site (e.g., the prostate), or a light source 33 such as a source of infrared, visible light or ultraviolet light to provide optical energy to the treatment probe.

The processor, controller and control electronics and circuitry can include one or more of many suitable components, such as one or more processor, one or more field-programmable gate array (FPGA), and one or more memory storage devices. In some embodiments, the control electronics controls the control panel of the graphic user interface (hereinafter “GUI”) to provide for pre-procedure planning according to user specified treatment parameters as well as to provide user control over the surgery procedure.

In some embodiments, the treatment probe 450 may comprise an anchor 24. The anchor 24 can anchor the distal end of the probe 450 while energy is delivered to energy delivery region 20 with the probe 450.

The probe 450 may comprise any suitable number of energy sources and configurations of energy sources. In some embodiments, the probe 450 may comprise an energy source 200 such as a nozzle, and the energy source 200 may comprise any suitable energy source as described herein such as a directional energy source that emits energy along a path in a direction for the selective treatment of tissue. Although reference is made to a water jet, the energy source 200 may comprise any energy source as described herein. Examples of suitable energy sources to treat tissue with a directional energy source such as a water jet are described in “PCT/US2015/048695, filed Sep. 5, 2015, entitled “PHYSICIAN CONTROLLED TISSUE RESECTION INTEGRATED WITH TREATMENT MAPPING OF TARGET ORGAN IMAGES”, published as WO2016037137, the full disclosure of which has been previously incorporated herein by reference.

In some embodiments, the energy source 200 is carried on a carrier 452, which is configured to translate and rotate the energy source as described herein. The energy source can be configured to move, e.g. with rotation and translation, while other components of the treatment probe 450 remain fixed.

The treatment probe 450 may be coupled to the first arm 442 with a linkage 430. The linkage 430 may comprise components to move energy delivery region 20 to a desired target location of the patient, for example, based on images of the patient. The linkage 430 may comprise a first portion 432, a second portion 434 and a third portion 436. The first portion 432 may comprise a substantially fixed anchoring portion. The substantially fixed anchoring portion 432 may be fixed to support 438. Support 438 may comprise a reference frame of linkage 430. Support 438 may comprise a rigid chassis or frame or housing to rigidly and stiffly couple the first arm 442 to treatment probe 450. The first portion 432 can remain substantially fixed, while the second portion 434 and third portion 436 can move to direct energy from the probe 450 to the patient. The first portion 432 may be fixed at a substantially constant distance 437 to the anchor 24. The substantially fixed distance 437 between the anchor 24 and the fixed first portion 432 of the linkage allows the treatment to be accurately placed. The first portion 432 may comprise a linear actuator to accurately position the second energy source such as high-pressure nozzle 200 in the energy delivery region 20 at a desired axial position along an elongate axis 451 of treatment probe 450.

The elongate axis 451 of treatment probe 450 generally extends between a proximal portion of the probe 450 near linkage 430 to a distal end having anchor 24 attached thereto. The third portion 436 can control a rotation angle 453 around the elongate axis 451. During treatment of the patient, a distance 439 between the energy delivery region 20 and the first portion 432 of the linkage may vary with reference to anchor 24. The distance 439 may adjust with translation 418 of the probe in response to computer control to set a target location along the elongate axis 451 of the treatment probe. In some embodiments, the first portion of the linkage remains fixed, while the second portion 434 adjusts the position of the energy delivery region 20 along the axis 451. The third portion 436 of the linkage adjusts the angle 453 around the axis in response to controller 424 such that the distance along the axis at an angle of the treatment can be controlled very accurately with reference to anchor 24. The probe 450 may comprise a stiff member such as a spine extending between support 438 and anchor 24 such that the distance from linkage 430 to anchor 24 remains substantially constant during the treatment. The treatment probe 450 is coupled to treatment components as described herein to allow treatment with one or more forms of energy such as mechanical energy from a jet, electrical energy from electrodes or optical energy from a light source such as a laser source. The light source may comprise infrared, visible light or ultraviolet light. The energy delivery region 20 can be moved under control of linkage 430 such as to deliver an intended form of energy to a target tissue of the patient.

The imaging console 490 may comprise a memory 493, communication circuitry 494 and processor 492. The processor 492 in corresponding circuitry is coupled to the imaging probe 460. An arm controller 491 is coupled to arm 444 to precisely position imaging probe 460. The imaging console may further comprise a display 425.

In order to facilitate precise control of the treatment probe and/or the imaging probe during treatment of the patient, one or more the treatment probe or the imaging probe may be coupled to a robotic, computer-controllable arm. For example, referring to system 400 shown in FIG. 2, one or both of the first arm 442 coupled to the treatment probe 450 as described herein and the second arm 444 coupled to the imaging probe 460 may comprise robotic, computer-controllable arms. The robotic arms may be operably coupled with one or more computing devices configured to control movement of the robotic arms. For example, the first robotic arm 442 may be operably coupled with the processor 423 of the console 420, or the second robotic arm 444 may be operably coupled with the processor 492 of the imaging console 490 and/or to the processor 423 of the console 420. The one or more computing devices, such as the processors 423 and 492, may comprise computer executable instructions for controlling movement of the one or more robotic arms. The first and second robotic arms may be substantially similar in construction and function, or they may be different to accommodate specific functional requirements for controlling movement of the treatment probe versus the imaging probe.

The robotic arm may comprise 6 or 7 or more joints to allow the arm to move under computer control. Suitable robotic arms are commercially available from several manufacturers such as RoboDK Inc., Kinova Inc., Kuka AG, and several other manufacturers.

The one or more computing devices operably coupled to the first and second robotic arms may be configured to automatically control the movement of the treatment probe and/or the imaging probe. For example, the robotic arms may be configured to automatically adjust the position and/or orientation of the treatment probe and/or imaging probe during treatment of the patient, according to one or more pre-programmed parameters. The robotic arms may be configured to automatically move the treatment probe and/or imaging probe along a pre-planned or programmed treatment or scanning profile, which may be stored on a memory of the one or more computing devices. Alternatively or additionally to automatic adjustment of the robotic arms, the one or more computing devices may be configured to control movement of the treatment probe and/or the imaging probe in response to user inputs, for example through a graphical user interface of the treatment apparatus. Alternatively or additionally to automatic adjustment of the robotic arms, the one or more computing devices may be configured to control movement of the treatment probe and/or the imaging probe in response to real-time positioning information, for example in response to anatomy recognized in one or more images captured by the imaging probe or other imaging source (from which allowable ranges of motion of the treatment probe and/or the imaging probe may be established) and/or position information of the treatment probe and/or imaging probe from one or more sensors coupled to the probes and/or robotic arms.

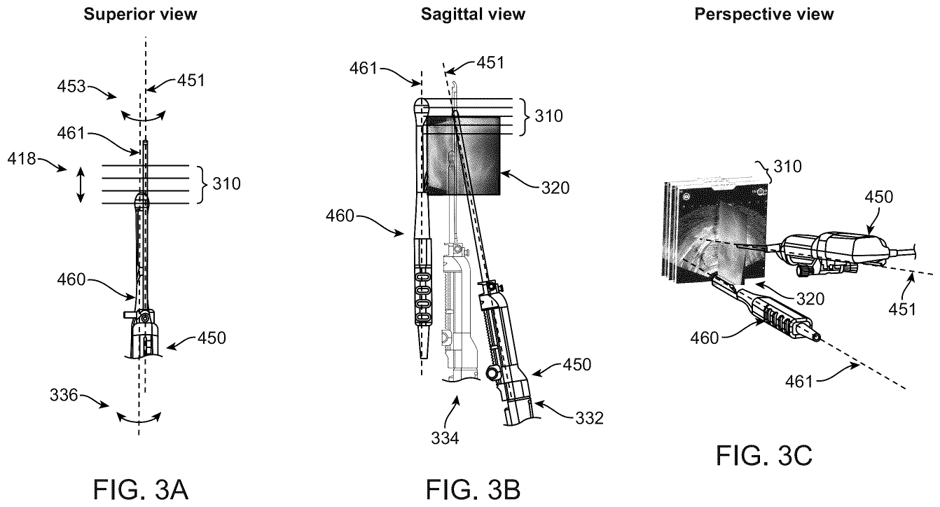

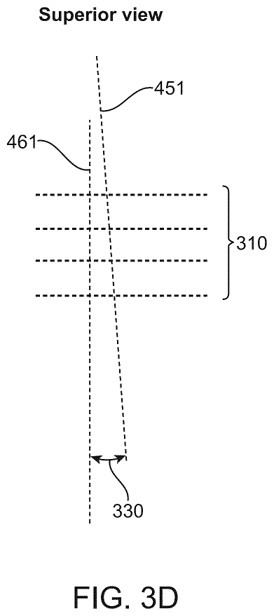

FIGS. 3A, 3B, and 3C show superior, longitudinal (e.g. sagittal), and perspective views, respectively, of an arrangement of probes for use in treatment of tissue. In particular, FIGS. 3A, 3B, and 3C show the relative arrangement, including position and orientation of a treatment probe 450 with respect to the position and orientation of an imaging probe 460 for the treatment of tissue such as prostate tissue. The imaging probe 460 can be configured to generate transverse images such as transverse ultrasound images 310 and one or more longitudinal images such as one or more longitudinal (e.g. sagittal) ultrasound images 320. In some embodiments, the energy source of treatment probe 450 is moved with rotation angle 453 and translation 418, such that the treated tissue and the energy source are within the field of view of the imaging probe 460.

As shown in the superior view of FIG. 3A, the treatment probe axis 451 and the imaging probe axis 461 are positioned in a substantially coplanar configuration, such that the imaging probe and the treatment probe extend along a common plane. As shown in the superior view of FIG. 3B and the perspective view of FIG. 3C, the treatment probe axis 451 and the imaging probe axis 461 are positioned in a substantially coplanar and non-parallel configuration, such that the imaging probe and the treatment probe substantially extend along a common plane, which allows the imaging probe to image the treatment probe along the length of translation 418 with one or more longitudinal images such as real time longitudinal images, for example real time sagittal images. In some embodiments, the treatment probe and the imaging probe are arranged in a substantially coplanar configuration, and the ultrasound probe is rotated to rotate the longitudinal (e.g. sagittal) field of view of the imaging probe in order to image the treatment probe along a length of the longitudinal field of view. Referring again to FIG. 3A, the imaging probe 460 can be rotated about elongate axis 461 by an angle 336 to align the treatment probe 450 so as to be within the longitudinal field of view, such that the longitudinal field of view of the imaging probe is aligned with elongate axis 451 of the treatment probe, for example.

One or more of the treatment probe or the imaging probe can be moved to adjust alignment between the imaging probe and the treatment probe. In some embodiments, the proximal portion of treatment probe is moved from a first position to a second position. Referring again to FIG. 3B, the treatment probe 450 can be moved from a first position 332 to a second position 334 to adjust alignment between the probes, for example.

In some embodiments, the imaging probe 460 and the treatment probe 450 are aligned to be substantially coplanar with each other within a margin of error so that the imaging probe 460 can image the treatment probe 450 and the treatment probe's energy source during treatment, for example with the treatment probe located within a field of view of the imaging probe such as a longitudinal (e.g. sagittal) image field of view. In some embodiments, the treatment probe is aligned with the imaging probe, such that the treatment probe is visible along a length of a longitudinal (e.g. sagittal) view of the imaging probe. Alternatively or in combination, the imaging probe can be oriented in with the sagittal plane field of view directed away from the treatment probe, in order to view an anterior region of treatment with the probes in an offset configuration as described hereon.

In some embodiments, the imaging probe 460 and the treatment probe 450 may be somewhat misaligned, e.g. by more than the margin of error, such that the treatment probe may disappear from a portion of the longitudinal (e.g. sagittal)) image because part of the imaging probe extends beyond the longitudinal (e.g. sagittal) field of view, for example. In some embodiments, this may result in the imaging probe 460 not imaging a portion of the treatment with longitudinal (e.g. sagittal) images. In some embodiments, the treatment probe 450 and the imaging probe 460 may be arranged in a substantially skewed orientation as described herein, e.g. outside the margin of error, such that the treatment probe extends outside the longitudinal (e.g. sagittal) field of view of the imaging probe but is located within the field of view of transverse images of the imaging probe. In such embodiments, the treatment can be monitored in real time with transverse images, in which the imaging probe moves to maintain the energy source and concurrently treated tissue within the transverse field of view of the imaging probe. In some embodiments, a transverse view of the tissue and energy source can decrease sensitivity to alignment between the two probes, and the imaging probe can be moved with the energy source, for example synchronously, to image the tissue and the energy source during treatment. In some embodiments, the ultrasound imaging probe is configured to translate with translation of the energy source to keep the energy source within the transverse field of view, for example with an offset configuration of the treatment probe and the imaging probe as described herein.

FIG. 3D shows the treatment probe axis 451 and the imaging probe axis 461 skewed with respect to each other by an angle 330, such that the treatment probe and the imaging probe do not extend along a common plane. By adjusting the treatment probe or the imaging probe, or both, this skew angle can be decreased. The amount of acceptable skew may depend on several factors such as the field of view of the imaging probe, the length of tissue treated with the one or more of rotating or translating energy source. In some embodiments, the margin of error of the skew angle is any one of no more than 10 degrees, no more than 5 degrees, no more than 3 degrees, no more than 2 degrees, or no more than 1 degree, for example. In some embodiments, the margin of error of alignment corresponds to a longitudinal (e.g. sagittal) field of view of the imaging probe and a skew angle between the imaging probe and the treatment probe. When the treatment probe and the imaging probe are aligned within the margin of error, the treatment probe is located within the longitudinal (e.g. sagittal) field of view along the translation length of the treatment and can be viewed in one or more real-time longitudinal (e.g. sagittal) images along a length of the longitudinal (e.g. sagittal) field of view. When the treatment probe and the imaging probe are aligned outside the margin of error, a portion of the treatment probe is located outside the longitudinal (e.g. sagittal) field of view and may disappear from a portion of an image along a length of the longitudinal (e.g. sagittal) field of view. In such embodiments, transverse imaging can be used to view the treatment in real time as described herein.

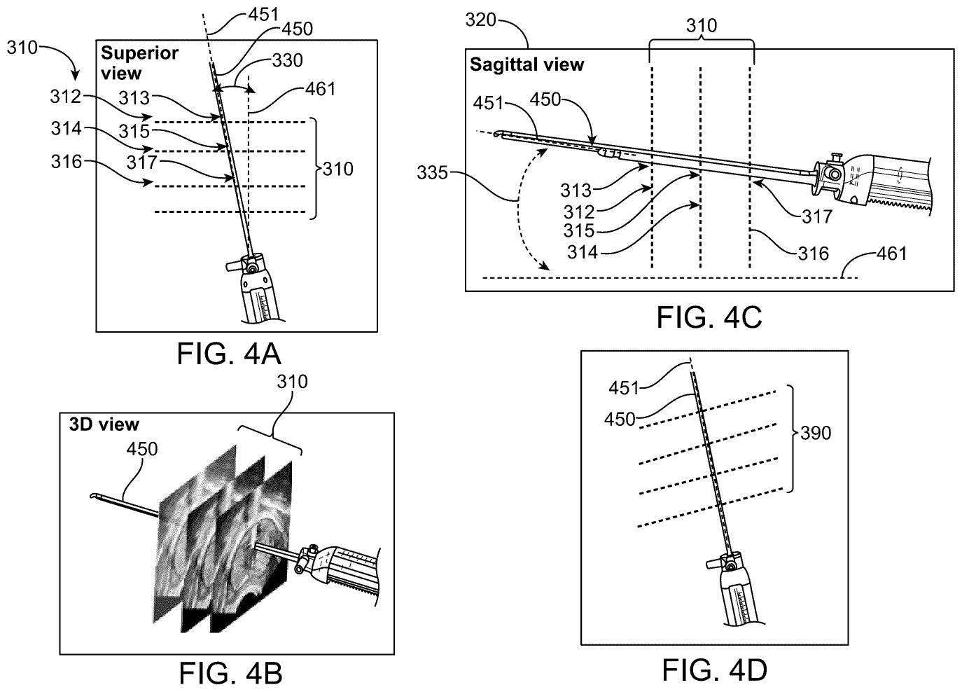

FIG. 4A shows a superior view of transverse image planes with respect to an axis of the treatment probe. In some embodiments, the axis 451 of treatment probe 450 comprises a non-coplanar orientation with respect to the elongate axis 461 of the imaging probe, for example with a skew angle 330. In some embodiments, the transverse images 310 comprise a plurality of transverse images such as a first transverse image 312, a second transverse image 314, and a third transverse image 316.

In some embodiments, the position of the treatment probe in the plurality of transverse images changes, for example with a lateral shift or a vertical shift, or a combination thereof. The extent to which the probe position changes can be related to a non-parallel angle such as skew angle 330 between the treatment probe and the imaging probe. In some embodiments, the treatment probe 450 extends through the plane of the first transverse image 312 at the first location 313, through the plane of the second transverse image 314 at the second location 315, and through the plane of the third transverse image 316 at the third location 317. In some embodiments, the skew angle 330 results in the lateral position of the treatment probe changing in the transverse images 330, such as the pixel column location of the transverse images.

FIG. 4B shows a three dimensional view of transverse images 310 and the corresponding planes of the transverse images with respect to treatment probe 450.

FIG. 4C shows the longitudinal (e.g. sagittal) image 320 of the treatment probe 450. In some embodiments, the axis 451 of the treatment probe 450 extends at an angle 335 with respect to the elongate axis 461 of the imaging probe. In some embodiments, the angle 335 corresponds to an angle of the axis 451 of the treatment probe 450 along the longitudinal (e.g. sagittal) plane of the image. This angle may result in in the treatment probe appearing inclined in the longitudinal (e.g. sagittal) image, the position of the probe 450 changing in the transverse images 310, for example. In some embodiments, the treatment probe is sufficiently coplanar with the imaging probe so as to be located within the longitudinal (e.g. sagittal) field of view of the imaging probe along a length of the longitudinal (e.g. sagittal) field of view, even though the position of the probe changes along longitudinal (e.g. sagittal) image 320 and the corresponding transverse images 310. In some embodiments, the angle 335 results in the vertical position of the treatment probe changing in the transverse images 310, such as a pixel row location in the transverse images. In some embodiments, the treatment probe 450 shown in the longitudinal (e.g. sagittal) image extends through the plane of the first transverse image 312 at the first location 313, through the plane of the second transverse image 314 at the second location 315, and through the plane of the third transverse image 316 at the third location 317. Although reference is made to the treatment probe at a skew angle with respect to the imaging probe, in some embodiments, the angle 335 corresponds to a 3D vector projection of the treatment probe axis onto the longitudinal (e.g. sagittal) image plane of the field of view, for example. In some embodiments, the skew angle 330 corresponds to a 3D vector projection of the treatment probe axis onto a plane that is perpendicular to the longitudinal (e.g. sagittal) image plane field of view.

In some embodiments, the position of the probe in the transverse images can be used to determine one or more of the three dimensional position or the three dimensional orientation of the treatment probe with respect to the imaging probe. In some embodiments, the two dimensional location of the probe 451 is determined in each of the plurality of transverse images, and these two dimensional locations are used to determine the three dimensional orientation of treatment probe with respect to the imaging probe. The two dimensional location of the probe in each transverse image may comprise any suitable two dimensional location such as X and Y locations or pixel locations such as pixel row location and a pixel column location in each image, for example. In some embodiments, the 3D orientation of the treatment probe with respect to the imaging probe comprises a 3D vector representation of the orientation, for example.

The three dimensional orientation of the treatment probe can be used to facilitate treatment planning, for example by generating rotated transverse images. The rotation of the image data set can also be used to generate one or more rotated longitudinal images, e.g. one or more rotated sagittal images. The one or more longitudinal images can be rotated such that the position of the treatment probe remains substantially fixed along the one or more longitudinal images and the plurality of transverse images. In some embodiments, the rotated longitudinal and transverse images are generated from a 3D tomographic image data set, such as a Digital Imaging and Communications in Medicine (DICOM) image data set, by selecting the planes of the plurality of transverse images and one or more longitudinal images by generating the images along planes that are oriented at angles relative to the planes X, Y and Z planes of the 3D tomographic image dataset.

FIG. 4D shows rotated transverse images 390, which correspond to the transverse images 310 rotated to compensate for the skew angle 330 between the elongate axis of the treatment probe and the imaging probe. In some embodiments, the three dimensional orientation of the treatment probe with respect to the imaging probe can be used to rotate the transverse images such that the rotated transverse image planes are substantially perpendicular to the elongate axis 451 of the treatment probe 450, for example to within one or more of 5 degrees of perpendicular, 3 degrees of perpendicular, 2 degrees of perpendicular, 1 degree of perpendicular, 0.5 degrees of perpendicular, or 0.25 degrees of perpendicular, for example. The one or more longitudinal images such as one or more sagittal images can be similarly rotated to compensate for the angle 335. In some embodiments, the transverse images 310 and one or more longitudinal images 320 comprise images of a 3D image data set, such as DICOM images. The 3D vector orientation of the treatment probe with respect to the imaging probe can be used to rotate the transverse and one or more longitudinal images, such that the location of the treatment probe remains substantially fixed in the transverse images, which can facilitate treatment planning as described herein. In embodiments the rotation of the 3D image data set results in the probe appearing at a substantially fixed elevation in the one or more longitudinal images such as one or more sagittal images, for example.

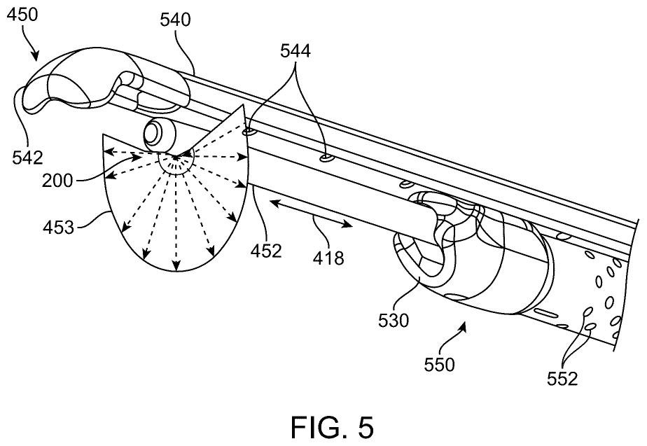

FIG. 5 shows treatment probe 450 and corresponding movements of an energy source 200. In some embodiments, the energy source 200 is carried on the carrier 452 that is configured to carry the energy source move the energy source as described herein. In some embodiments, the carrier 452 is configured to carry the energy source to a plurality of angles and longitudinal positions corresponding to a treatment plan such as a three dimensional treatment plan as described herein. In some embodiments, energy source 200 is configured to rotate to a plurality of angles with rotation of carrier 452 in accordance an angle 453 of the treatment plan as described herein. In some embodiments, the energy source is configured to translate with translation of carrier 452 as shown with arrows 418. The rotational movement and translational movement of the energy source can be combined in accordance with the 3D treatment plan as described herein, for example.

The energy source 200 may comprise any suitable energy source, such as such as one or more of an electrode, a loop electrode, a laser source, a thermal energy source, a mechanical energy source, a mechanical sheer, an ultrasound probe, a cavitating ultrasound probe, a water jet, e.g. a fixed pressure water jet, a plasma source, a steam source, a morcellator, a trans urethral needle, a photo ablation source, a radiation energy source, a microwave energy source or a water jet evacuation source, for example.

While the treatment probe 450 can be configured in many ways, in some embodiments the treatment probe comprises an opening 530 configured to receive an endoscope such as a cystoscope to view the treatment area. In some embodiments, the probe 150 comprises a support portion 550 that is configured to support the carrier and the endoscope. The support portion 550 may comprise one or more openings 552 configured to release a fluid such as saline from a saline source such as an elevated bag of saline. In some embodiments, the probe comprises a distal portion 540 comprising sufficient stiffness to advance the probe into the patient. In some embodiments, the stiff portion 540 extends to a curved distal end 542 on the tip of the probe 450 to allow the probe to be inserted into tissue, such as along a lumen. In some embodiments, the stiff portion 540 comprises one or more openings 544 to remove material such as fluids from the surgical site. In some embodiments, the one or more openings 544 is coupled to an evacuation pump such as the aspiration pump as described herein.

While the probe 450 can be configured in many ways, in some embodiments, the carrier 452 is configured to move the energy source 450 while other components of the probe remain fixed, such as the distal portion 540. Work in relation to the present disclosure suggests that the distal portion 540 can help to stabilize tissue while the energy source such as a water jet treats the tissue. In some embodiments, the one or more openings 544 to remove material are located distal to the one or more openings 552 that release fluid such as saline in order to promote fluid flow past the opening 530 that receives the endoscope in order to flush material such as resection products away from the viewing port of the endoscope to improve visibility of the tissue treated with the endoscope. In some embodiments, the one or more openings that remove material from the surgical site are located distal to the endoscope opening and at least some positions of the energy source during treatment in order to draw tissue treatment products away from the endoscope viewing port.

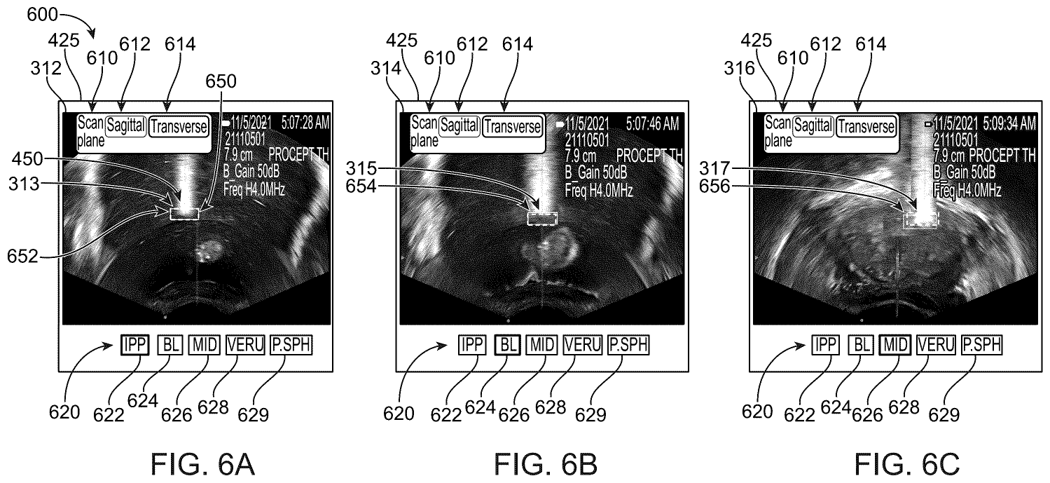

FIGS. 6A to 6C show a user interface 600 and transverse images that show movement of the probe location in the transverse images, which can be related to orientation of the treatment probe with respect to the imaging probe. FIG. 6A shows the transverse image 312 and the location 313 of the treatment probe 450 in the image. FIG. 6B shows the transverse image 314 and the location 315 of the treatment probe in the image. FIG. 6C shows the transverse image 316 and the location 317 of the treatment probe in the image. As will be apparent in the images, the location of the probe changes in the transverse images. The location of the probe in the transverse images can be used to determine the orientation of the treatment probe with respect to the imaging probe as described herein.

In some embodiments, a marker 650 is used to identify the location of the probe in one or more transverse images. In some embodiments, a marker is shown on each of a plurality of transverse images. In some embodiments, a first marker 652 is shown at a first location of a first transverse image, such as location 313 of the transverse image 312, a second marker 654 is shown at a second location of a second transverse image such as location 315 of transverse image 314, and third marker 656 is shown at a third location of a third transverse image such as location 317 of transverse image 316.

In some embodiments, an artificial intelligence algorithm is configured to identify the location of the probe and identify the probe with the marker that corresponds to the location of the probe. The marker may comprise any suitable marker, such as a line, mark, a series of marks, a reticle, a cross, or a geometric shape such as a triangle or polygon, e.g. a box. In some embodiments, the user interface is configured to allow the user to adjust the position of the marker, for example after reviewing an initial position determined with the algorithm as described herein.

In some embodiments, a user interface 600 is configured to show the images on the display 425 in response to user inputs. In some embodiments, user interface 600 is configured to display a scan plane 610 with an associated user input 612 for the user to select one or more longitudinal (e.g. sagittal) plane images and an associated user input 614 for the user to select the transverse images. In some embodiments, the user interface 600 comprises a plurality of user selectable inputs 620 for the user to select a transverse image for viewing on the display. The plurality of user selectable inputs 620 may comprise a separate input for each plane, such as a user selectable button, tab, or pull down menu, for example. In some embodiments, a first user selectable input 622 corresponds to a first transverse image 312 along a first plane, a second user selectable input 624 corresponds to a second transverse image 314 along a second plane, a third user selectable input 626 corresponds to a third transverse image 316 along a third plane, a fourth user selectable input 628 corresponds to a fourth transverse image along a fourth plane, and a fifth user selectable input 629 corresponds to a fifth transverse image along a fifth plane.

In some embodiments, the first user input 622 corresponds to an intravesical prostatic protrusion (IPP), the second user input 624 corresponds to the bladder neck (BL), the third user input 626 corresponds to the mid prostate (MID), the fourth user input 628 corresponds to the verumontanum (VERU), and the fifth user input 629 corresponds to the peripheral sphincter (P. SPH). Although reference is made to anatomical landmarks of the prostate, the user selectable inputs may correspond to any structure visible in the image, such as any anatomical structure of any organ, or structure of a probe such as a treatment probe, for example.

Although reference is made to five user inputs and five corresponding transverse images along corresponding planes, the number of inputs and corresponding images may comprise any suitable number, such as two user selectable inputs corresponding to a first transverse image and a second transverse image. Alternatively, more than five user selectable inputs and transverse images can be used.

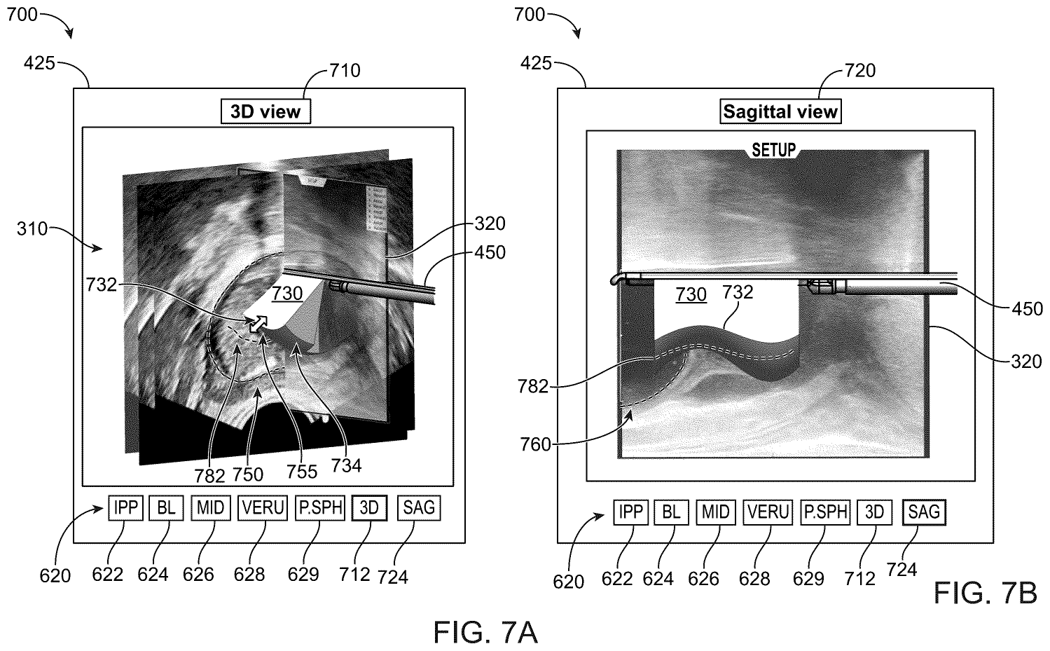

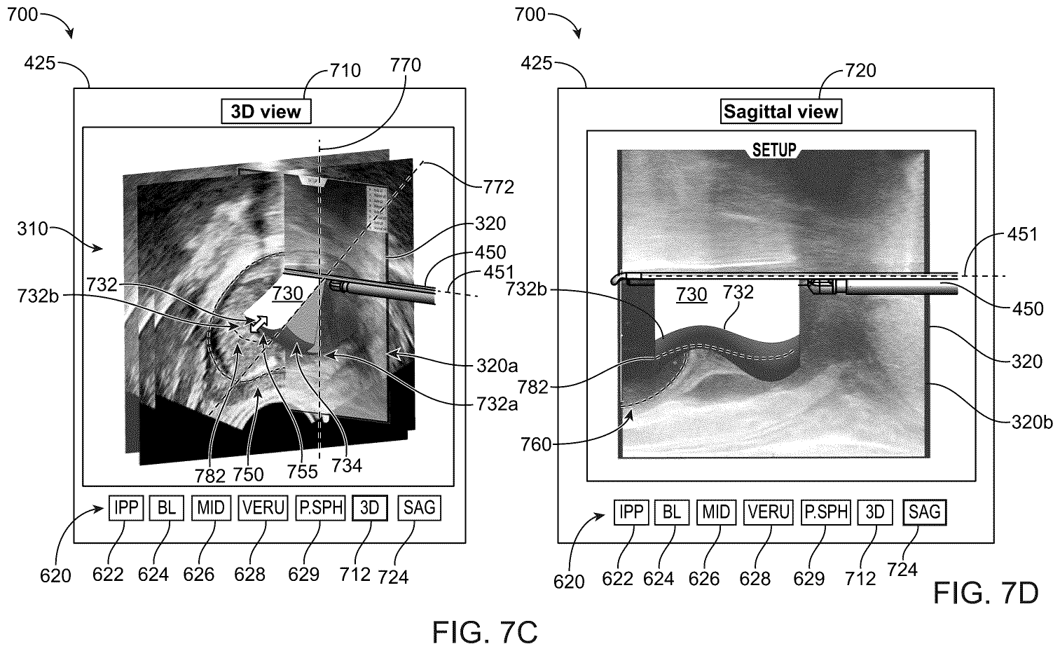

FIG. 7A shows a user interface 700 with a three dimensional view 710 for three dimensional treatment planning. In some embodiments, an animation of the treatment probe 450 is overlaid on a plurality of 2D images such as 2D ultrasound images, which are arranged in a 3D digital environment, which allows view the images with a 3D perspective. In some embodiments, the user interface is configured for the user to select an image among the plurality of images to maximize the selected image with another view. The plurality of 2D images may comprise a plurality of transverse images 310, which are arranged along one or more longitudinal images such as one or more sagittal images 320. In some embodiments, each of the plurality of transverse images 310 is located along the one or more longitudinal images such as one or more sagittal images 320 at a position corresponding to an intersection of the transverse image and the one or more longitudinal (e.g. sagittal) images, such as an intersection in a 3D data set. In some embodiments, the user interface 700 is configured for a user to select an image to maximize a view of the image, for example by clicking on the image, and provide an enlarged view of the image. For example, the user can select one or more of the longitudinal (e.g. sagittal) images or a transverse image to view the selected image with an enlarged view.

In some embodiments, an AI algorithm is used to identify tissue structures and develop a 3D treatment plan, such as a 3D treatment profile as described herein.

The treatment profile 730 may comprise an animated treatment profile, which allows the user to view the treatment profile from different perspectives, angles and images, for example. In some embodiments, the user is provided with a control to one or more of zoom, pan, or rotate the perspective view. In some embodiments, the treatment profile 730 comprises an animated treatment profile that matches the one or more of the zoom, pan or rotation of the perspective view, such that alignment of the treatment profile with the plurality of images is maintained. This approach can allow the user to view the treatment profile and its relationship with corresponding tissue from any suitable perspective. In some embodiments, the animated treatment profile is configured to generate simulation of the treatment with movement of the energy source and an increasing volume of treatment tissue, such similar to movie that simulates the treatment.

In some embodiments, the user interface 700 is configured for the user to adjust the treatment profile, and the treatment profile is automatically updated and shown on the perspective view and other selected images, for example simultaneously updated in real time. The user interface may comprise an input 755 such as a visible icon that the user can drag to change the position of the treatment profile. In some embodiments, the user interface 700 is configured with an input 755 such as a 3D input for the user to adjust the treatment profile shown in the 3D view. In some embodiments, when the treatment profile is adjusted in one of the views with the user input, the treatment profile is automatically updated in the other views, for example simultaneously updated, so that the user can evaluate the change in the treatment profile from more than one perspective. In some embodiments, the treatment profile comprises a plurality of curved lines, such as splines, which are updated together and shown on the different views.

FIG. 7B shows a longitudinal view (e.g. sagittal) view 720 for three dimensional treatment planning. In some embodiments, a longitudinal treatment profile such as a sagittal treatment profile 732 is overlaid on one of the one or more longitudinal images such as on one of the one or more sagittal images 320. While the user interface 700 can be configured in many ways, in some embodiments the user is presented with longitudinal view 720 and a 3D perspective view 710, for example. In some embodiments, the user interface is configured for the user to select the image from the one or more images longitudinal (e.g. sagittal) images 320 from the perspective view and provide the longitudinal (e.g. sagittal) view of the image as shown in FIG. 7B.

In some embodiments, the 3D treatment profile 730 is updated in response to the user input such as a 3D user input, and the corresponding sagittal treatment profile 732 and transverse treatment profile 734 of the 3D treatment profile updated in the corresponding views such as the longitudinal and transverse views as described herein. For example, the longitudinal (e.g. sagittal) treatment profile 732 can be updated with user input 755 on the 3D view 710 to move the longitudinal treatment profile 732 from a first longitudinal treatment profile to a second longitudinal treatment profile 782, and the updated treatment profile will be shown in the other views, such as the longitudinal (e.g. sagittal) view as shown in FIG. 7B. Similarly, the 3D treatment profile 730 can be adjusted in the longitudinal (e.g. sagittal) view 720 from a first longitudinal treatment profile 732 to a second longitudinal (e.g. sagittal) treatment profile 782 and the updated treatment profile shown in the 3D view 710. The treatment profiles in the transverse views can be similarly adjusted updated and shown in the 3D view and longitudinal (e.g. sagittal) views, for example.

The one or more longitudinal images 320 shown in the 3D view can be configured in many ways and may comprise any suitable number of longitudinal images, such as a single longitudinal image or a plurality of longitudinal images, for example. The one or more longitudinal images may comprise one or more sagittal or parasagittal images, for example. In some embodiments, the user interface 700 is configured to receive a user input identifying a selected image among the one or more longitudinal images 320 and the plurality of transverse images shown in the 3D view and display the selected image with a 2D view.

In some embodiments, the one or more longitudinal images comprises a plurality of longitudinal images. In some embodiments, the user interface 700 is configured for the user to select one or more longitudinal images 320 from a plurality of longitudinal images. In some embodiments, the user interface 700 is configured for a user to select a longitudinal image in the 3D view. For example, the user interface 700 can be configured for the user to select the longitudinal image with a pointing device our touching the image on a touch screen display as described herein. Alternatively or in combination, the plurality of inputs 620 may comprise a plurality of inputs corresponding to a plurality of longitudinal images. Alternatively or in combination the user input 724 may comprise a plurality of inputs configured for a user to select a plurality of longitudinal images for display in a plurality of 2D views.

While the plurality of longitudinal images can be generated in many ways, in some embodiments the plurality of longitudinal images has been generated from a 3D volumetric image of the tissue as described herein.

In some embodiments the plurality of longitudinal images has been generated in response to a plurality of angles of an energy source to treat the tissue. In some embodiments, the 3D treatment profile 730 corresponds to a plurality of rotations and translations of the energy source on the probe 450 as described herein, and at least one of the plurality of longitudinal angles corresponds to a rotation angle of the energy source.

FIGS. 7C and 7D show a plurality of rotational angles relative to the treatment probe that can be used to generate a plurality of longitudinal images. The plurality of longitudinal images may comprise longitudinal images corresponding to rotation angles the energy source of the treatment probe 450 about axis 451. For example, 3D view may comprise a first longitudinal image 320a of the one or more images 320 corresponding to a first rotational angle 770 of the energy source, and a second longitudinal image 320b corresponding to a second rotational angle 772 of the energy source as shown in FIG. 7B.

In some embodiments, plurality of longitudinal images comprises a first longitudinal image 320a along a first portion 732a of the treatment profile and a second longitudinal image 320b along a second portion 732b of the treatment profile.

In some embodiments, the arrangement in the 3D view shows a portion of the first longitudinal image along the first portion of the treatment profile and a portion of the second longitudinal image along the second portion of the treatment profile. In some embodiments, the image 320b is placed in the 3D view 710 at the angle 772, and the image 320a is also shown in the 3D view 710 at the angle 770. The user can select which longitudinal images to show in the 3D view with the user interface as described herein. The plurality of longitudinal images may comprise any suitable number of images and may comprise at least 3 longitudinal images, each at a different angle of rotation with respect to an elongate axis of a treatment probe.

In some embodiments, the first longitudinal image comprises a first transparency along the first portion of the treatment profile and a second transparency along the second portion of the treatment profile greater than the first transparency to increase visibility of the second longitudinal image along the second portion. In some embodiments, the second longitudinal image comprises a first transparency along the first portion of the treatment profile and a second transparency the second portion of the treatment profile, the first transparency greater than the second transparency to increase visibility of the first longitudinal image along the first portion of the treatment profile.

In some embodiments, an AI algorithm is used to process the image data and identify one or more anatomical tissue structures and provide mark on the one or more anatomical tissue structures in one or more of the views shown on the user interface, such as on a longitudinal (e.g. sagittal) view and a transverse view. In some embodiments, a mark such as mark 750 is shown on the one or more of the transverse images 310 of the 3D perspective view, and the mark 750 can be shown on the corresponding transverse view. In some embodiments, a mark such as mark 760 corresponding to one or more anatomical tissue structures is shown on the one or more longitudinal views 720 of one or more longitudinal images such as one or more sagittal images 320, for example. The mark 750 and the mark 760 may generally comprise any suitable change to the pixels overlaid on the image, such as one or more of highlighting, dashes, lines, icons or other features so as to indicate the profile identified with the AI algorithm.

In some embodiments, a plurality of views is shown simultaneously on the user interface, for example the 3D perspective view of FIG. 7A and the one or more longitudinal views of FIG. 7B. Alternatively or in combination, the 3D perspective view can be shown with one or more transverse views. The plurality of views can be arranged in any suitable way, for example a side by side configuration, or a view inside a view configuration, for example. In some embodiments, each of the user selectable views is shown in a pop up window that the user can one or more of move, resize, or close, for example.

In some embodiments, the user interface 700 comprises an input 712 for the user to select the three dimensional view and an input 724 for a user to select one or more longitudinal views such as one or more sagittal views 720, and a treatment profile such as a 3D treatment profile 730 is overlaid on the images. In some embodiments, the 3D treatment profile comprises a plurality of transverse treatment profiles 734 and one or more longitudinal (e.g. sagittal) profiles 732, which can be overlaid on the corresponding images. The user interface 700 may comprise one or more features of user interface 600, for example.

In some embodiments, the images shown in user interface 700 comprise rotated images such as images from a rotated 3D image, in which the images have been rotated in response to an orientation between the imaging and treatment probe as described herein. Alternatively, the images may comprise unrotated images, such as unrotated 3D images, for example.

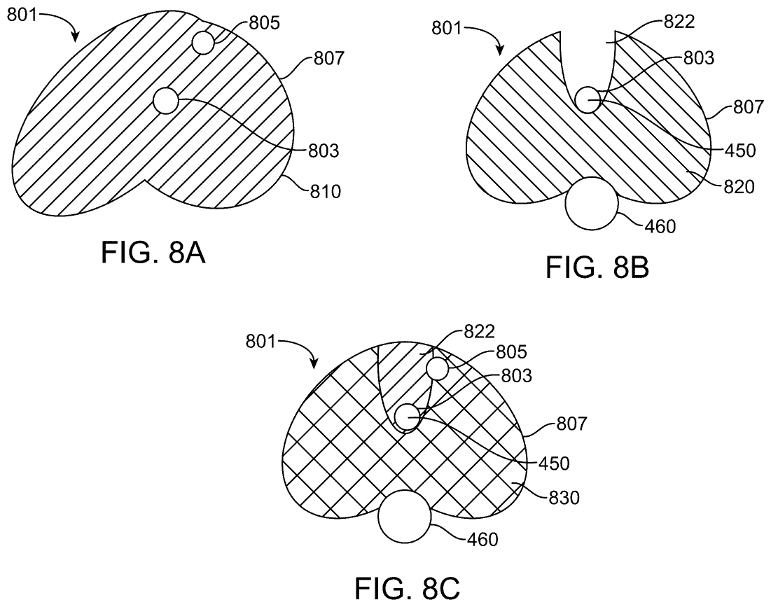

FIG. 8A shows a diagnostic image 810 to detect cancer, in accordance with some embodiments. The diagnostic image may comprise any suitable image, such as a tomogram from any suitable imaging device such as one or more of an X-ray image; a fluoroscopic image, a computed tomography (CT) image; a magnetic resonance imaging (MRI) image; positron emission tomography (PET) image, single-photon emission computed tomography (SPECT) image. In some embodiments, the diagnostic image shows a lesion 805, or other image data consistent with cancerous tissue. In some embodiments, the diagnostic image comprises an MRI image of a prostate 801 with a lesion 805. Although any suitable tissue can be imaged, in some embodiments, the diagnostic image 810 comprises an image of a prostate 801 and associated prostatic tissue such as a urethra 803, a capsule 807.

FIG. 8B shows a configuration of a treatment probe an imaging probe for generating an intra operative image such as an ultrasound image 820, in accordance with some embodiments. Although many intraoperative imaging devices can be used in accordance with the present disclosure, in some embodiments the image 820 comprises a TRUS probe image, for example. The treatment probe 450 is visible in the image and may be placed in the urethra 803, for example. The treatment probe 450 generates a shadow 822 in the ultrasound image 820 such as a TRUS image. The position of the TRUS probe 460 and treatment probe 450 are shown. The lesion 805 as shown in FIG. 8A may correspond to a location inside the shadow 822 or a location outside of the shadow 822 and combinations thereof in the ultrasound image of FIG. 8B. In some embodiments, the lesion 805 is identifiable in the diagnostic image and is not identifiable in the ultrasound image 820, although the lesion 805 is visible in the fused image, which can facilitate treatment planning as described herein. In some embodiments, the lesion 805 is located outside of the shadow 822 and is not visible in the intraoperative image 820 because the lesion is not detectable with the ultrasound imaging probe. In some embodiments, the lesion 805 is located within the shadow 822 and is not visible in the image 820 because the lesion is within the shadow and is not detectable with the ultrasound imaging probe. Alternatively or in combination, the lesion may be visible in the ultrasound image and the diagnostic image, for example.

FIG. 8C shows a fused image 830, in accordance with some embodiments. The fused image can be generated in many ways in response to common landmarks between the diagnostic image 810 and the intraoperative image 820, such as a with deformable image fusion, for example. The landmarks may comprise any suitable landmarks, such as one or more of the urethra, the bladder neck, the mid prostate, the verumontanum, the prostate capsule and ducts, for example. The fused image 830 may comprise any of the tissue structures shown in the diagnostic image 810 and the intraoperative image 820, e.g. ultrasound image, such as one or more of an organ, a prostate 801, a lesion 805, a lumen such as urethra 803, a capsule 806, a treatment probe 450, or a shadow 822, for example. In some embodiments, the fused image 830 shows the lesion 805, which is not present in the intraoperative image 820, as described herein with reference to FIG. 8B, for example. In some embodiments, the lesion 805 appears at a location outside shadow 822 in the fused image 830. In some embodiments, the lesion 805 appears at a location within shadow 822 in the fused image 830. In some embodiments, the lesion 805 extends from a location outside shadow 822 to a location inside shadow 822 in the fused image 830.

The fused image 830 can be configured in many ways, and may comprise a 3D volumetric image, such as a DICOM image for example, which is fused with the real time ultrasound image and overlaid with the treatment plan. In some embodiments, the fused image comprises a layer, e.g. a plane, from the diagnostic image corresponding to the location of the real time ultrasound image. In some embodiments, the fused image data comprises a heatmap of cancer probability, for example. In some embodiments, the fused image data comprises data such as segmented data indicating a tissue type such as median lobe of the prostate, a capsule of the prostate, a urethra, etc. In some embodiments, the user interface is configured with a plurality of user selectable inputs to display a type of data, such as a heat map, overlaid on the fused image.

Although reference is made to specific tissue types and probabilities, in some embodiments the fused image comprises data related to the durability of tissue. For example, some types of tissue with lower amounts of collagen can be more easily resected with the energy source, e.g. the water jet, while other types of tissue with greater amounts of collagen are more resistant to the water jet. In some embodiments, the displayed data may show an indicator related to durability of the tissue, such a “glandularness”, corresponding to tissue that is less resistant to removal, and “stromalness” which corresponds to tissue which is more difficult to remove such as the collagenous capsule of the prostate. In some embodiments, the energy source such as a water jet is located at a distance from the capsule, such that the softer cancerous tissue is removed from the capsule and the collagenous capsule remains intact.

In some embodiments, the fused image is remapped after removal of a portion of the tissue such as a portion of the prostate and prior to treating a second portion of the tissue such as the prostate.

In some embodiments, the fused image 830 comprises data from other inputs. In some embodiments, the treatment probe comprises an endoscope that is used to identify tissue. The endoscope can be advanced and retracted with the probe placed in the patient. In some embodiments, the probe tip is configured to contact tissue, and a user selection indicates that the probe location corresponds to a selected tissue such as a verumontanum of the prostate or other tissue. In some embodiments, the endoscope is located on a carriage with sensors such as encoders, and the position of the endoscope can be used to determine the location of tissue, for example when the distal tip of the probe or the endoscope contacts the tissue. Alternatively or in combination, the probe may comprise impedance sensors, for example. In some embodiments, a position sensor such as a coil or magnetic element is used to contact and label tissue, which is shown in the fused image, for example.

In some embodiments, electromagnetic (EM) sensors are used to determine the position of one or more of the treatment probe 450 or the imaging probe 460, and the positions used to determine the position of the one or more probes with respect to the fused image 830 as described herein, for example with reference to FIG. 2.

In some embodiments, a phantom probe such as a polymer phantom probe, e.g. silicone or plastic, is placed in one or more of the urethra or the rectum of the patient when the diagnostic image 810 is generated, to decrease differences between the diagnostic image 810 and the ultrasound image 820. The dimensions and placement of the phantom probes can be similar to the treatment probe 450 and imaging probe 460, for example.

FIGS. 7A to 7B show treatment planning and treatment on fused images 630. Alternatively or in combination, the treatment planning and profile can be performed on a real time ultrasound images such as a real time transverse image or longitudinal image (e.g. sagittal image), for example. In some embodiments, the treatment is performed as a series of passes with the treatment probe at one or more of a different location or a different orientation for each of the passes. In some embodiments, each of the plurality of passes corresponds to a 3D volumetric treatment.

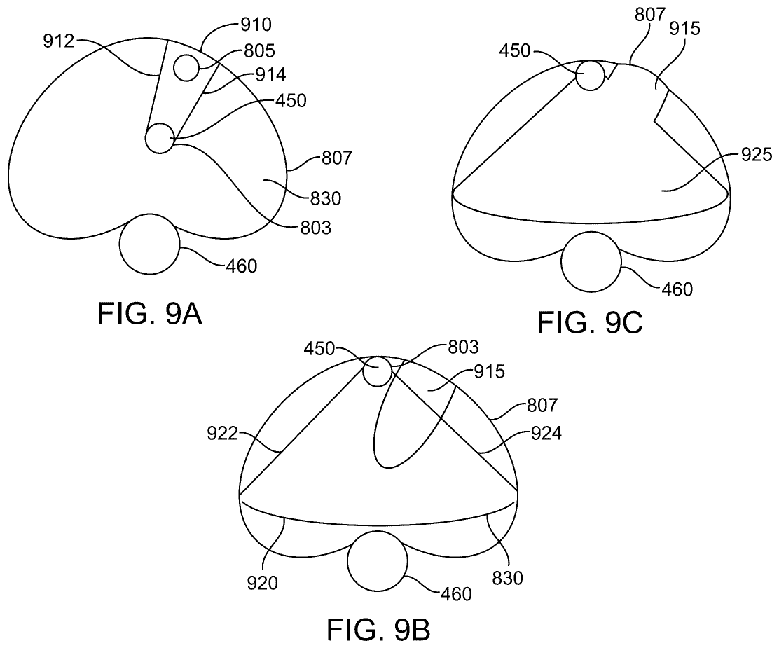

FIG. 9A shows the fused image 830 and a first treatment plan to treat a lesion, in accordance with some embodiments. In some embodiments, the first treatment plan comprises a first angle 912 of treatment, a second angle of treatment 914, and a profile 910 corresponding to a depth of resection from the treatment probe between angle 912 and angle 914, with treatment probe 450 place in a lumen such as urethra 803. In some embodiments, the treatment plan corresponds to a profile 910 of tissue resection, which may comprise a three dimensional 3D tissue resection profile. The profile 910 corresponds to a first treatment angle 912 of the energy source and a second treatment angle 914 of the energy source such as a water jet. The energy source is configured to rotate between the first angle and the second angle so as to treat the tissue with the energy source to a described depth, e.g. to sweep the energy source. Once the energy source has completed treatment along a planned profile of a first transverse plane, the energy source can be translated to perform the treatment along another transverse plane, and additional planes so as to treat the tissue along a 3D treatment profile.

In some embodiments, the energy source is configured to selectively remove a first tissue type such as glandular tissue, e.g. cancerous tissue, while leaving a second tissue type such as collagenous tissue, e.g. tissue of capsule 807. In some embodiments, the energy source comprises a water jet configured to kill cancerous cells adjacent a prostate capsule, e.g. by lysing the cells, without removing tissue of the capsule 807. In some embodiments, the energy source such as a water jet is configured to kill cancerous cells embedded within or on the surface of the prostate capsule.

FIG. 9B shows a second treatment profile 920 to treat a second region of tissue and a second treatment plan to treat a second rejection of tissue after the first region has been treated and one or more probes repositioned, in accordance with some embodiments. As shown in FIG. 9B, the resected tissue from the treatment plan of FIG. 9A produces a tissue void 915. While the treatment can be performed in many ways, in some embodiments the treatment probe is coupled to a source of saline, such as a constant pressure source, e.g. a hung bag of saline, which at least partially fills void 915 with a fluid such as saline. As shown in FIG. 9B, in some embodiments the capsule 807 remains substantially intact adjacent void and at least partially defines void 915. In some embodiments, the power (e.g. flow rate) and movement of the energy source (e.g. rotational speed) are configured to treat the capsule 807 without perforating the capsule. In some embodiments, the energy source is repeatedly swept across the capsule 807 at the location of the void 915 to ensure that the cancerous cells have been removed from the region of capsule 807 that defines void 915 without penetrating capsule 807 with the energy source such as the water jet.

In some embodiments, the treatment probe 450 is initially positioned so as to treat anterior tissue of the prostate as shown in FIG. 9A, and then repositioned so as to treat a different region of the prostate. Because the tissue of the prostate can be somewhat soft, the treatment probe 450 can be repositioned so as to tent the prostate tissue.

In some embodiments, the second treatment plan comprises a first angle 922 of the second treatment, a second angle of treatment 924, and a profile 920 corresponding to a depth of resection from the treatment probe between angle 922 and angle 924, with treatment probe 450 placed in a lumen such as urethra 803. In some embodiments, the second treatment plan corresponds to a second treatment profile 920, which is defined by a first treatment angle 922 and a second treatment angle 924.

FIG. 9C shows tissue after completion of the first treatment and the second treatment, in accordance with some embodiments. These treatments result in first void 915 and second void 925. In some embodiments, the first void and the second void overlap to form a continuous void, for example.

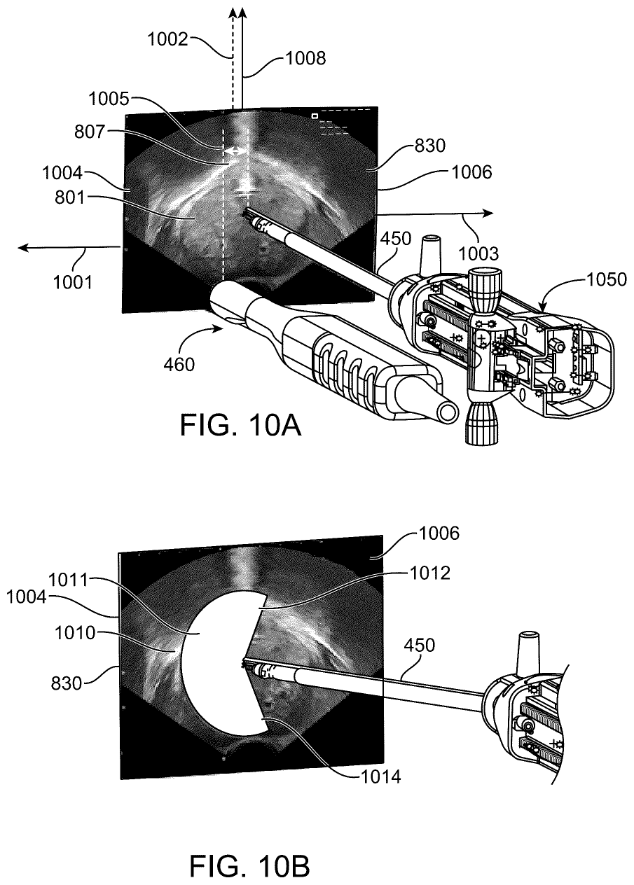

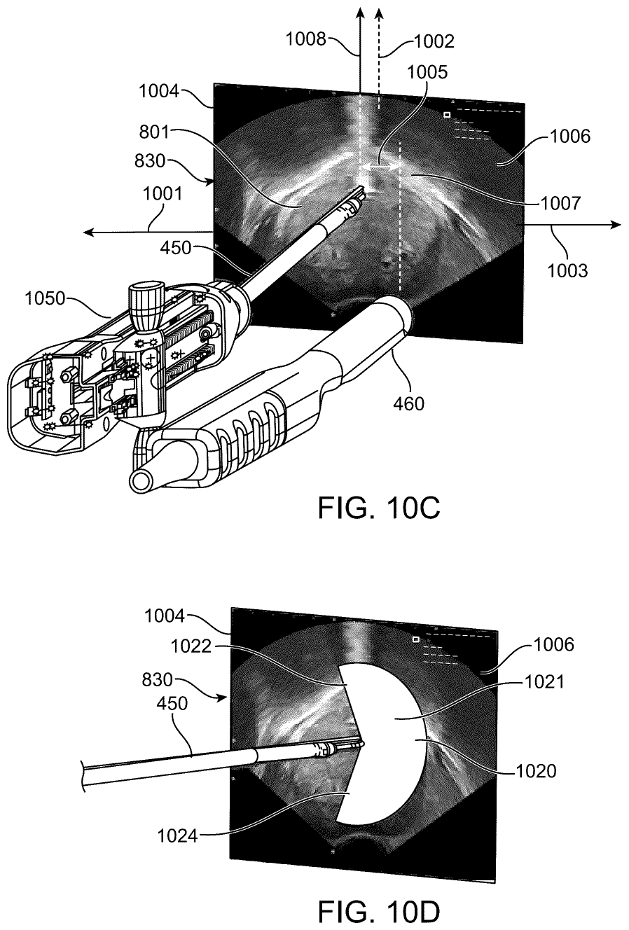

FIGS. 10A and 10B show a first configuration of a treatment probe 450 and an imaging probe 460 to treat tissue on a first side 1004 of the patient, e.g. Setup A directed to treating tissue on a right side of the patient, in accordance with some embodiments. The anterior direction 1008 is shown with an arrow. The imaging probe 460 is located posteriorly, e.g. in a rectum of a patient, and the treatment probe 450 is located anterior to the imaging probe, e.g. in a urethra of a patient. In some embodiments, the patient comprises a midline 1002, which extends in an anterior posterior direction, so as to define a first side 1004 on a first side of the patient, e.g. the patient's right side 1001, and a second side 1006 on a second side of the patient, e.g. on the patients left side 1003. In some embodiments, the midline 1002 of the patient defines a boundary between the first side 1004 and the second side 1006. The offset 1005 between the treatment probe and the imaging probe can allow the imaging probe to image anterior prostate tissue with decreased interference from the treatment probe, e.g. a decreased shadow or other artifact caused by the presence of the treatment probe 450 in the imaging field. In some embodiments, the fused image 830 is shown on the display as described herein. Alternatively or in combination, ultrasound images from probe 460 can be used to position the probes, for example.

In some embodiments, one or more of the treatment probe or the imaging probe are arranged with an offset 1005 relative to the midline 1002 to allow the imaging probe 460 to view anterior tissue, such as anterior tissue of the capsule 807 of the prostate 801. Work in relation to the present disclosure suggests that cancerous tissue can grow near an anterior region of the prostate 801, e.g. near the anterior capsule 1007, and the offset 1005 can be helpful to image this tissue. While the offset 1005 can be provided in many ways, in some embodiments, the treatment probe 450 is moved to the side of the midline while the imaging probe 460 remains along the midline 1002. Alternatively or in combination, imaging probe 460 can be offset from the midline.

As shown in FIG. 10A, the treatment probe 450 is shown rotated so that the energy source faces toward the first side 1004 of the patient with the treatment probe 450 placed on the second side 1006 of the patient with respect to the midline. Alternatively, the imaging probe 460 can be placed on one side of the midline 1002, e.g. on the first side 1004 in order to image tissue anterior to the treatment probe 450.

FIG. 10B shows a first sweep angle 1010 and targetable areas 1011 with the first configuration, e.g. Setup A, in accordance with some embodiments. In some embodiments, the probe is configured to sweep between an anteriorly directed angle 1012 and a posteriorly directed angle 1014. In some embodiments, the sweep angle 1010 is defined by the angle between the angle 1012 and the angle 1014. With treatment planning software and markers, the user is able to provide inputs to adjust the sweep angle and distance of treatment from the probe with rotation of the energy source about the longitudinal axis of treatment probe 450. In some embodiments, the treatment planning is performed on the fused image 830 to direct treatment toward cancerous tissue or suspected cancerous tissue in response to the diagnostic data on the fused image.

FIG. 10C shows a second configuration of the treatment probe and the imaging probe of FIG. 10A to treat tissue on a second side 1006 of the patient, e.g. to treat the left side 1003 of the patient, with Setup B, in accordance with some embodiments. With the second configuration the offset 1005 has changed to an opposite configuration in order to treat an opposite side of the patient. The treatment probe 450 is shown rotated so that the energy source faces toward the second side 1006 of the patient with the treatment probe 450 placed on the first side 1004 of the patient with respect to the midline 1002. Alternatively, the imaging probe 460 can be placed on one side of the midline 1002, e.g. on the second side 1006, in order to image tissue anterior to the treatment probe 450.

While the first configuration of FIG. 10A and the second configuration of FIG. 10B can be provided in many ways, in some embodiments, the treatment probe 450 is positioned on a first side 1004 of the midline in the first configuration and a second side 1006 of the midline 1004 in the second configuration. In some embodiments, a location of the imaging probe 460 such as the TRUS probe remains at a substantially fixed location for the first configuration and the second configuration and the treatment probe 450 is moved from the first side in the first configuration to the second side in the second configuration. In some embodiments, the portion of the imaging probe 460 inserted into the patient such as the TRUS probe extends along the midline of the patient in the first configuration and the second configuration. In some embodiments, the portion of the treatment probe 450 inserted into the patient does not extend across the midline of the patient in the first configuration with the first offset and the portion of the treatment probe inserted into the patient does not extend across the midline in the second configuration with the second offset. In some embodiments, the first configuration as shown in FIG. 10A comprises a first offset 1005, and the second configuration shown in FIG. 10C comprises a second offset 1005 opposite the first offset. The first and second offsets can be provided in many ways, for example with movement such as translation of one or more of the treatment probe or the imaging probe, or a combination thereof, for example.

Alternatively or in addition to moving, e.g. translating one or more of the treatment probe 450 or the imaging probe 460, a handpiece 1050 of the treatment probe 450 can be rotated between the first configuration and the second configuration. In some embodiments, the handpiece is rotated 180 degrees between the first configuration and the second configuration. In some embodiments, the handpiece 1050 comprises internal linkages to rotate and translate the energy source to in response to motion control signals from a processor or controller as described herein. In some embodiments, the handpiece 1050 is configured to couple to a motor-pack (not shown), in which the motor control pack comprises motors to drive the linkages of the handpiece. In some embodiments, one or more of the EM coil sensors as described herein are provided on the motor-pack, which is non-sterile and reusable, and the position of the motor-pack is used to determine the position of the treatment probe in the fused images based on the known dimensions of the treatment probe. Alternatively or in combination, one or more of the EM sensors can be located on the imaging probe 460, for example on a proximal location of the imaging probe such as the TRUS probe, such that the one or more EM sensors is not inserted into the patient, for example.

FIG. 10D shows a sweep angle 1020 and targetable areas 1021 with a second sweep angle of the probe in the second configuration, in accordance with some embodiments. In some embodiments, the probe is configured to sweep between an anteriorly directed angle 1022 and a posteriorly directed angle 1024. In some embodiments, the sweep angle 1020 is defined by the angle between the angle 1022 and the angle 1024. With treatment planning software and markers, the user is able to provide inputs to adjust the sweep angle and distance of treatment from the probe with rotation of the energy source about the longitudinal axis of treatment probe 450. In some embodiments, the treatment planning is performed on the fused image 830 to direct treatment toward cancerous tissue or suspected cancerous tissue in response to the diagnostic data on the fused image.

While the sweep angle 1010, targetable areas 1011, the sweep angle 1020, and the targetable areas 1021 can be configured in many ways, in some embodiments the sweep angles and targetable areas are configured to overlap.

In some embodiments, a processor is configured to receive an input corresponding to an orientation of the handpiece the treatment probe, and the processor is configured to determine a corresponding position and rotational angle of the energy source relative to the handpiece to treat the targeted areas in response to the orientation of the handpiece.

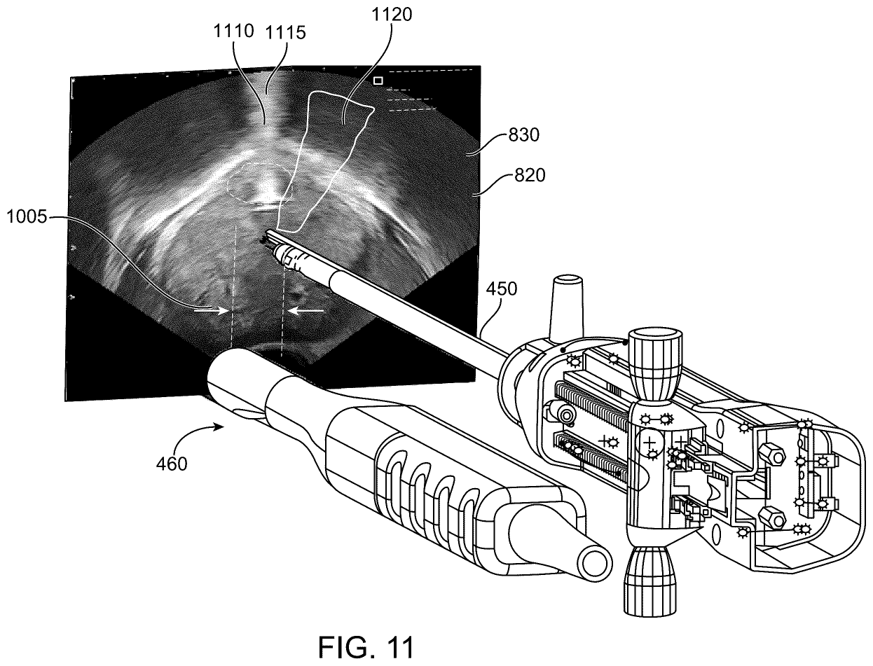

FIG. 11 shows an arrangement of a treatment probe 450 and an imaging probe 460 with an offset 1005 between the treatment probe and the imaging probe, in accordance with some embodiments. The offset 1005 can be provided by offsetting one or more of a treatment probe or an imaging probe as described herein. In order to decrease the effect of the ultrasound shadow caused by the handpiece 450, the treatment probe 450 or the TRUS probe 460 can be offset to effectively “see around” the treatment probe 450. When targeting areas on patient right, Tx probe 450 is offset to Pt left or TRUS 460 is offset to patient right to position ultrasound shadow optimally outside of the area of interest, and vice versa for targeting areas on patient left.

The area of interest 1110 comprises an area that would be helpful to visualize with the intraoperative image such as a TRUS image. However, with a first configuration of the treatment probe 450 and imaging probe 460, a shadow 1115 of the treatment probe is produced at the region of interest 1110. By offsetting the treatment probe 450 in relation to imaging probe 460, the ultrasound shadow 1120 occurs at a location away from the region of interest 1110, such that the region of interest can be imaged without shadow 1115 overlapping with and obscuring the region of interest 1110.

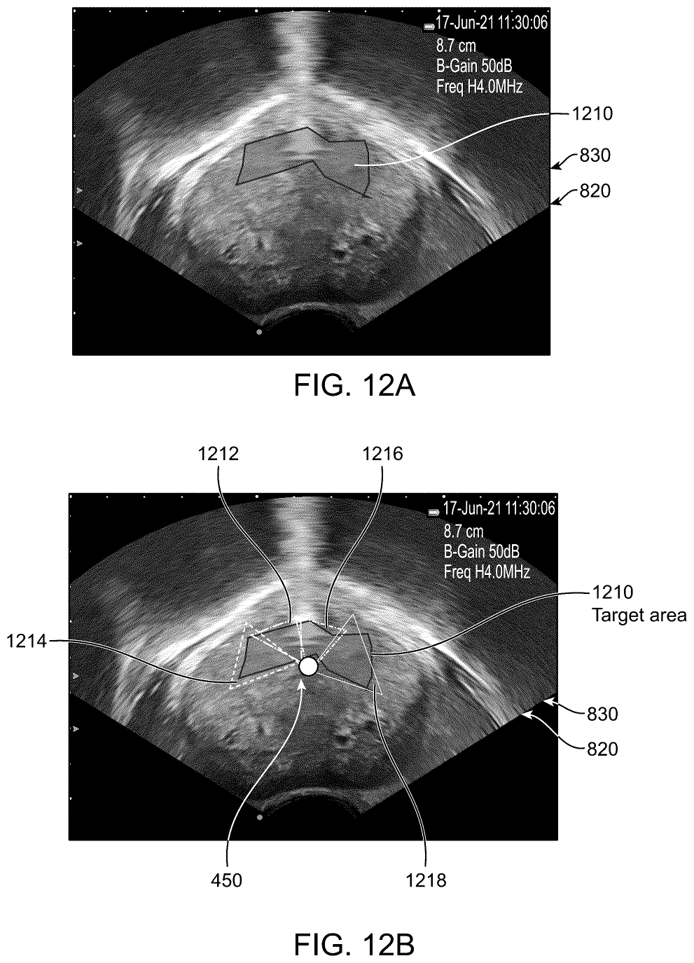

FIGS. 12A and 12B shows treatment planning and treatment of an anterior lesion, in accordance with some embodiments. In some embodiments, the tissue to be treated is divided into a plurality of treatment zones.

FIG. 12A shows a targeted treatment area 1210, in accordance with some embodiments. The target area 1210 may be determined at least in part based on diagnostic data from fused image 830 and may include lesions from a diagnostic image, for example.

FIG. 12B shows tissue treatment regions with the first configuration of the probes and the second configuration of the probes shown in FIGS. 10A to 10D, in accordance with some embodiments. The treatment planning can be performed with user inputs to a treatment profile overlaid on the fused image 830. In some embodiments, the treatment planning is performed with a plurality of user adjustable inputs on a touch screen display. In some embodiments, the treatment plan comprises a plurality of user adjustable regions to be resected in sequence and corresponding to the configuration of the treatment probe and the imaging probe, e.g. in accordance with the first configuration or the second configuration.

Treatment planning for treating an anterior lesion may comprises the following steps, which may be performed in sequence: to treat a plurality of treatment zones. 1) with the handpiece and treatment probe 450 oriented in a first configuration as shown with reference to FIGS. 10A and 10B, e.g. Setup A to treat the patient's right, a first resection 1212 is planned, e.g. cut #1. In some embodiments, a small treatment angle of the first resection allows more effective visualization of the area by seeing around the treatment zone. With the probes in the first configuration a second resection 1214 is planned, e.g. cut #2. Second resection 1214 is performed for any lateral target areas, which may be less prone to cancer. One or more of the treatment probe or the imaging probe is moved to provide an offset, e.g. translated, and the handpiece rotated e.g. with 180 degree rotation of the handpiece, to perform additional tissue resection with the second configuration as shown with reference to FIGS. 10C and 10D. A third resection 1216 such as cut #3 is planned with the probes arranged in the second configuration, which may be directed to cancerous tissue in response to diagnostic data on fused image 830, similarly to the first resection. A fourth resection 1218 such as cut #4 can be planned in response to data on the fused image, similarly to the second resection. In some embodiments, the treatment zones can be arranged to overlap in order to increase effective treatment coverage with the energy source.

The treatments as described with reference to FIGS. 8A to 12B may use any suitable energy source as described herein, and in some embodiments the energy source comprises a water jet.

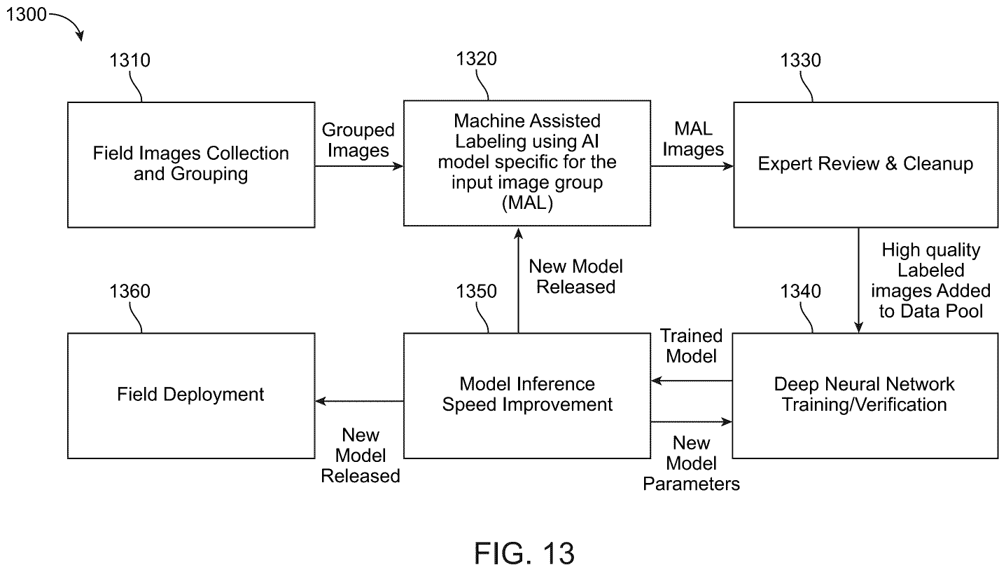

FIG. 13 shows a method 1300 of training an AI algorithm.

At a step 1310, images are collected and grouped. The images may comprise a collection of images acquired prior to and during treatment. The images can be appropriately classified and grouped, for example classified and grouped with respect to a type of tissue. In some embodiments, the images are group according to a type of tissue, such as types of prostate tissue as described herein, for example.

At a step 1320, the AI model receives the grouped images from step 1310. The machine assisted labeling (MAL) is used to is used to label the grouped images in accordance with each group of the images. In some embodiments, each group of images may be segmented and classified and labeled with a MAL. In some embodiments, the images are segmented and annotated with labels so as to identify tissue structures as described herein, for example.

At a step 1330, the MAL images are received by a user interface that allows an expert to review and cleanup of the MAL image data. The initial set of MAL images may be reviewed an expert such as a radiologist, for example. The expert review and cleanup of the MAL images generates high quality labeled image data. The high quality labeled image data can be added to a pool of high quality image data. This high quality image data can be used as a ground state or truth state to further train and refine the classifier and contains annotated data with appropriate labels identifying the type of tissue.

At a step 1340, the high quality labeled data is received by an AI algorithm as described herein and used for training and validation of the model. The annotated images can be used to train and validate the AI algorithm and develop model parameters of the AI algorithm. The AI algorithm may comprise any suitable algorithm as described herein, such as a neural network, e.g. a deep neural network for example.

At a step 1350, the trained model parameters generated at step 1340 are received to refine and tune the model. While this can be performed in many ways, in some embodiments model inference speed improvements are performed on the model to increase the speed of the model without substantially compromising the output of the model. This can be helpful to increase throughput of the model and decrease processing bottle necks in the model.

At a step 1360, the model is released and deployed in the field. This field deployed model can be used to process images to generate one or more tissue structures as described herein.

In some embodiments, the model is further refined prior to the field deployment at step 1360. For example, it can be helpful to iterate and refine the model by repeating steps 1320, 1330, 1340 and 1350 to generate acceptable model parameters for field deployment at step 1360. In some embodiments, steps 1320, 1330, 1340 and 1350 comprise elements of a feedback loop. In some embodiments, the new model parameters developed at step 1350 are provided to the grouped images for MAL at step 1320 and then MAL images provided to the expert for review at step 1330. In some embodiments, additional images are provided for testing and validation at step 1320 and MAL images generated and provided to the expert for review at step 1330, and these images added to the pool of image data. In some embodiments, the new model parameters generated at step 1350 can be provided to the AI algorithm at step 1340 and used to evaluate the images and used to further refine and develop the AI algorithm at step 1340. Once the AI algorithm training and development has been completed at step 1340, this trained model can be refined at step 1350 for model inference speed improvement, for example. The steps, 1320, 1330, 1340 and 1350 can be performed as many times as appropriate to further refine and improve the model prior to field deployment at step 1360.

Although a method 1300 of training an AI algorithm is shown and described in accordance with some embodiments, one of ordinary skill in the art will recognize many adaptations and variations in accordance with the present disclosure. For example, the steps can be performed in any order. Some of the steps repeated, and some of the steps omitted. Some of the steps may comprise sub steps of other steps. Also, one or more of the steps of method 1300 can be combined with any step of any method described herein.

The processor as described herein can be configured to perform one or more steps of any method disclosed herein, such as method 1000, method 1100, method 1200 or method 1300, for example.

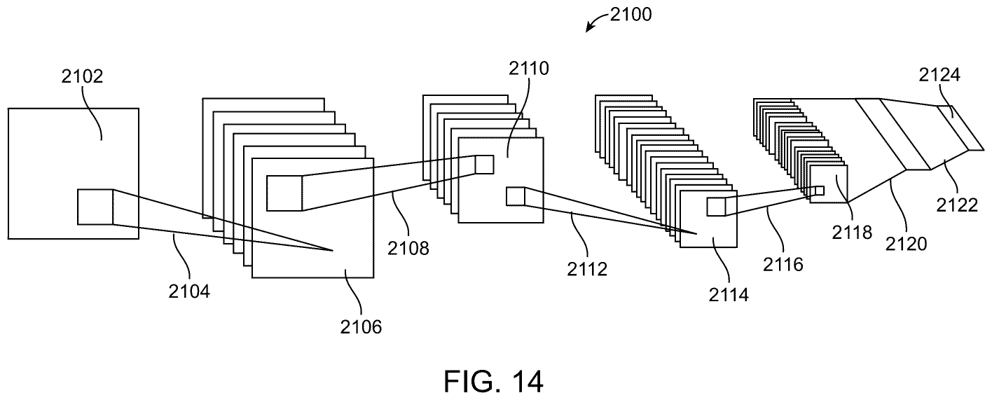

FIG. 14 shows an artificial intelligence (“AI”) algorithm suitable for incorporation in accordance with embodiments of the present disclosure. In some embodiments, the artificial intelligence algorithm comprises one or more of image enhancement, image segmentation, a neural network, a convolutional neural network, a transformer, a transformer machine learning model, supervised machine learning, unsupervised machine learning, edge detection, feature recognition, segmentation, 3D model reconstruction, or a multi-modality image fusion, for example.

In some embodiments, the AI algorithm comprises a two-dimensional convolutional neural network (CNN) 2100. In some embodiments, the AI such as a CNN is configured to identify one or more tissue structures of one or more tissue and process the images identify the tissue structure and determine a response of the tissue to treatment. The tissue may comprise a first tissue, or a second tissue, or combinations thereof as described herein. A dataset 2102 is initially provided, which may include imagery from historical treatment data from prior patients and procedures. A convolution operation 2104 results in data in a 2nd data set 2106, which in turn has a pooling layer 2108 applied to result in a pooled layer 2110 of subsample data in order to further condense the spatial size of the representation. The subsample data may be convoluted 2112 to produce a third data set 2114, which may further have a pooling layer applied 2116 to provide subsample data 2118. The subsample data 2118 may be passed through a first fully connected layer 2120 and a second fully connected layer 2122 to generate a classification matrix output 2124. One or more filters can be applied at each convolution layer to provide different types of feature extraction. After the model is defined, it may be compiled and may utilize accuracy of the feature recognition as a performance metric. The model may be trained over time, such as by using historical procedure data as training data and verified according to the model's predictions and verified over time until the model's predictions converge with truth data.

While the trained model can be configured in many ways, in some embodiments the trained model is configured to identify a tissue structure and output one or more metrics associated with the tissue structure, such as one or more of shape data or movement data as described herein.

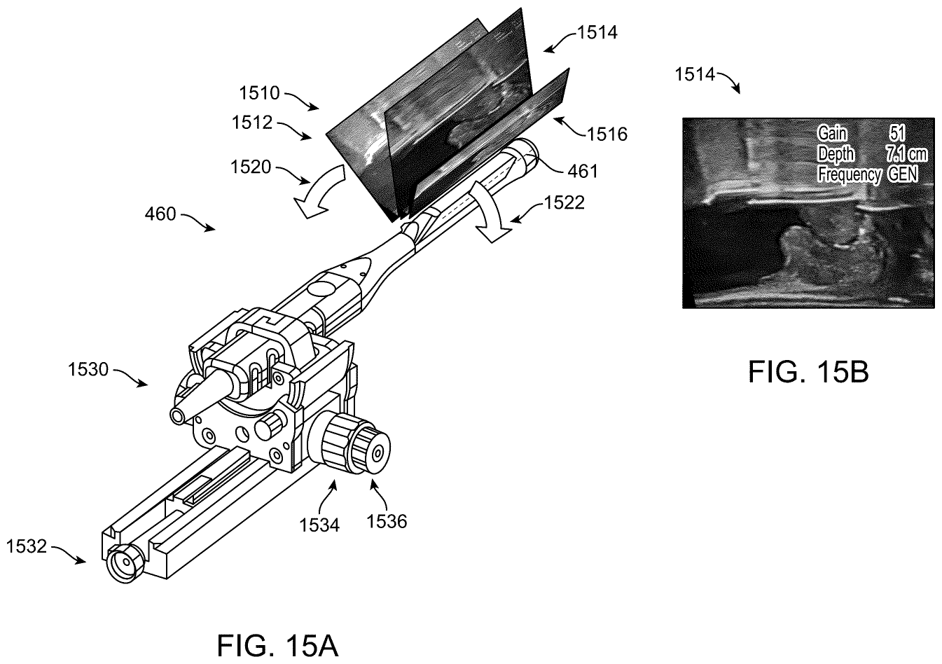

FIG. 15A shows rotation of an imaging probe 460 such as a TRUS probe about a longitudinal axis to acquire a plurality of longitudinal images 1510, in accordance with some embodiments. The plurality of longitudinal images 1510 may comprise a first longitudinal image 1512, a second longitudinal image 1514 and a third longitudinal image 1516. In some embodiments, second longitudinal image 1514 comprises a sagittal image, for example. In some embodiments, the first longitudinal image 1512 corresponds to rotation of the imaging probe 460 about the longitudinal axis in a first direction 1520 and third longitudinal image 1516 corresponds to rotation of the imaging probe 460 in a second direction 1522 opposite the first direction 1520. In some embodiments the first direction 1520 corresponds to a counterclockwise rotation and the second direction 1522 corresponds to a clockwise rotation, when viewed from the proximal end of the imaging probe 460 corresponding to the user's perspective.

While the imaging probe 460 can be supported in many ways, in some embodiments, the imaging probe 460 is supported with linkage 1530 configured to provide rotation of the imaging probe 460 in the first direction and the second direction. Alternatively or in combination, the linkage 1530 can be configured to advance and retract the imaging probe 460. In some embodiments, the linage 1530 comprises one or more knobs to allow the user to rotated and translate the probe. Alternatively or in combination, a motorized linkage can be used to rotate and translate the imaging probe 460. The linkage 1530 can be supported on an arm as described herein.

FIG. 15B shows a longitudinal image 1514 among the plurality of longitudinal images 1510 of FIG. 15A, in accordance with some embodiments. In some embodiments, the longitudinal image 1514 comprises a sagittal image, for example.

Referring again to FIGS. 1 and 2, when the imaging probe 460 is coupled to a lockable arm 444, the coupling apparatus may include any suitable structures to provide translational and rotational movement of the imaging probe after insertion into the patient (e.g., a stepper). The suitable structures may comprise one or more mechanisms, linkages, gears, motors, belts, pulleys, wires, or chains capable of providing the movement. These movements may comprise manual or motor controlled movements or a combination thereof. These movements may be calibrated and measurable, e.g. with electronic sensos, to measure and provide precise positioning changes of the imaging probe. In some embodiments, this movement is motorized and may be automated. In some embodiments, the user interface comprises one or more inputs that allow the user such as a surgeon to control this movement and measure positions of the imaging probe.

Alternatively or in combination, the movement of the imaging probe can be directed by an artificial intelligence algorithm. The algorithm may directly control a motors and control circuitry, or provide instructions on the user interface for the surgeon to control the motors and linkage to position the imaging probe.

In some embodiments, the calibrated positioning of the imaging probe allows a plurality of images, e.g. a plurality of image slices, of the desired anatomy to be captured at precise measurement points to allow creation of a 3D representation of the target anatomy. The 3D representation may comprise any suitable representation and may be generated by any suitable algorithm.

In some embodiments, the imaging probe is rotated around its longitudinal axis to acquire a plurality of longitudinal images, e.g. a plurality of longitudinal slices. In some embodiments, the number of rotational or translational positions at which images are acquired can be related to the fidelity of a 3D image and corresponding model reconstructed from the individual images, e.g. individual image slices. When the number of positions corresponds to the resolution of the imaging probe, a highly representative 3D image and corresponding model may be generated.

In some embodiments, the positioning of the imaging probe is performed manually by the user such as a surgeon. The user interface may be configured to provide guidance to the user as to speed of rotation or translation of the imaging probe. This guidance promotes sufficient time for the system to capture each image, e.g. image slice, before moving onto the next position. While the guidance can be configured in many ways, in some embodiments the guidance comprises color scales (e.g., green for ability to capture each slice before moving onto the next), bar graphs, speedometers, audible signals, tactile feedback on the rotational element, haptic resistance or other types of feedback as will be known to one of ordinary skill in the art.

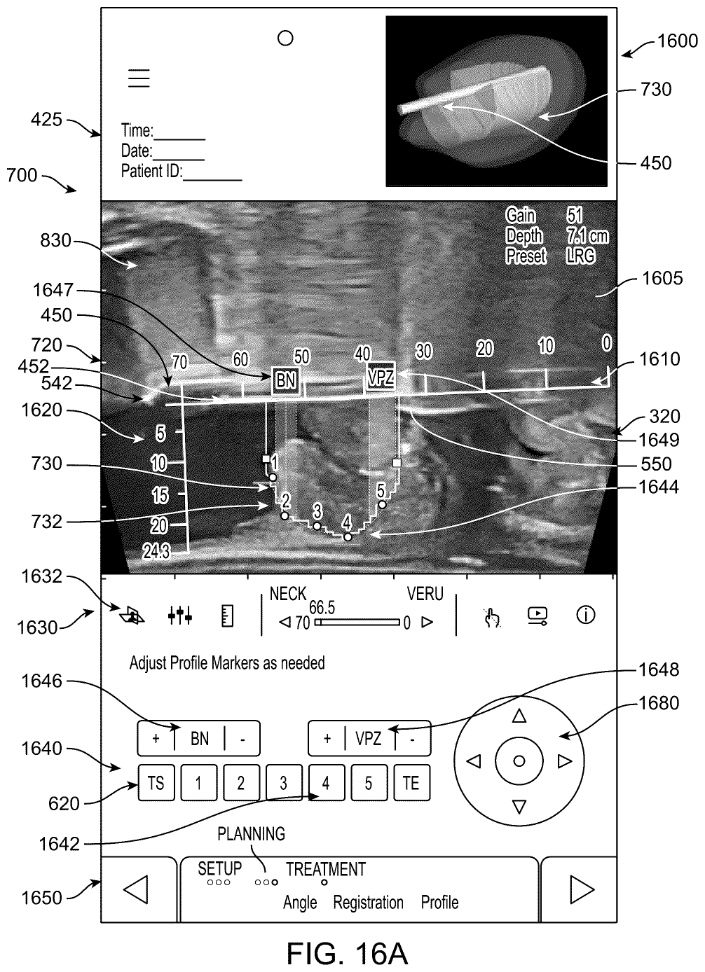

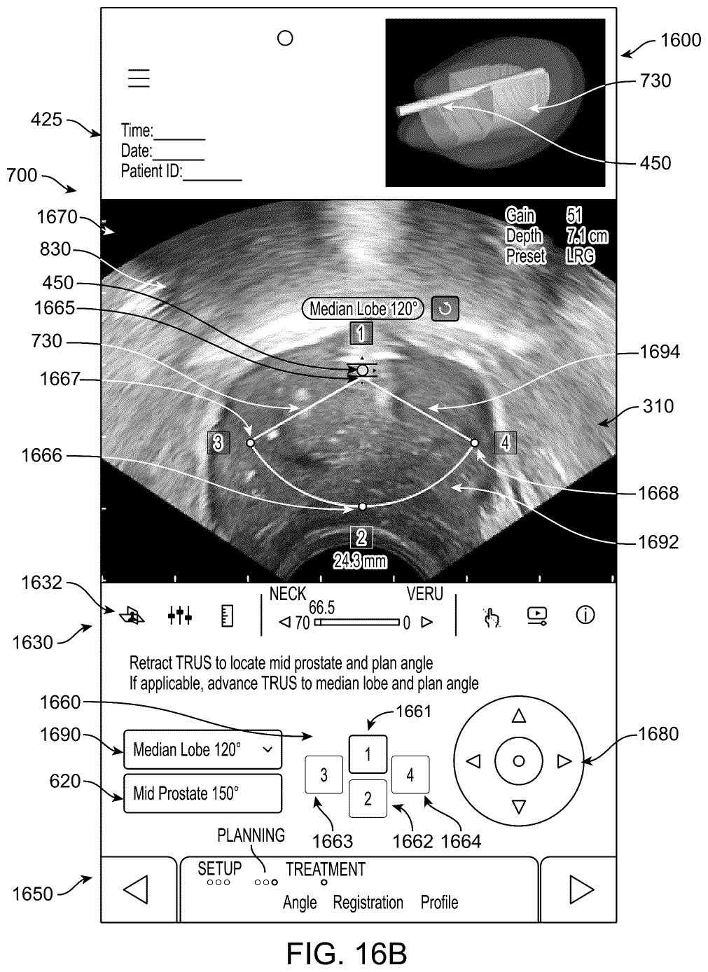

FIG. 16A shows user interface 700 on a display 425 with a 3D treatment profile 730 viewed in a longitudinal view 720 such as a sagittal image 320 and viewed in a 3D perspective view 1600, in accordance with some embodiments. The image 320 may comprise any suitable image as described herein, such as one or more longitudinal (e.g. sagittal) ultrasound images 320. In some embodiments, the 3D treatment profile 730 comprises a longitudinal image profile such as a sagittal image profile 732. In some embodiments, the treatment profile is overlaid on the fused image 830. In some embodiments, a real time longitudinal image 1605 has a plurality of treatment planning markers overlaid thereon. In some embodiments, the real time longitudinal image 1605 comprises a fused image 830, in which the pre-operative diagnostic image is fused with a real time ultrasound image in real time, e.g. with a latency of no more than one second.

The one or more images 320 may comprises one or more real time images, such as a real time longitudinal ultrasound image, e.g. a sagittal image. In some embodiments, one or more structures of the treatment probe 450 are visible in the image 320, such as the curved distal end 542 on the tip of the probe 450, the distal portion 540 of the probe 450, the carrier 452, support portion 550 with the endoscope placed in opening 530 to view tissue, for example as described herein with reference to FIG. 5.

Referring again to FIG. 16A, in some embodiments the position of the verumontanum is input by the user on the intraoperative image Alternatively or in combination, the position of the bladder neck (BN) is input by the user on the intraoperative ultrasound image. The intraoperative image may comprise an ultrasound image, such as a longitudinal image, e.g. sagittal image from a TRUS probe.

The position of one or more delicate tissue structures, such as the bladder neck (BN, NECK) and verumontanum (Veru, VERU) can be input by the user. In some embodiments, the user interface comprises an input 1646 configured for a user to adjust a location of a marker 1647 corresponding to a location of the bladder neck on the image 320. The input 1646 is configured for the user to move the bladder neck marker 1647 proximally and distally along image 320. In some embodiments, the bladder neck marker 1647 is configured to move along longitudinally extending ruler 1610. Alternatively or in combination, the directional pad 1680 can be configured to receive a user input to position the bladder neck marker 1647 directly over the bladder neck. In some embodiments, the position of the verumontanum corresponds to the location of the distal end of the support 550 as viewed in image 320.

In some embodiments, the user interface comprises an input 1648 configured for a user to adjust a location of a marker 1649 corresponding to a location of the verumontanum on the image 320. The input 1648 is configured for the user to move the VERU marker 1649 proximally and distally along image 320. In some embodiments, the VERU marker 1649 is configured to move along longitudinally extending ruler 1610. Alternatively or in combination, the directional pad 1680 can be configured to receive a user input to position the VERU marker 1649 directly over the VERU.

Although reference is made to the user determining the location of marker 1647 and marker 1649, in some embodiments AI used to determine the location of the bladder neck and veru and position the markers on the display accordingly. In some embodiments, the user interface is configured for the user to adjust the locations of these markers on the display after being placed in response to the locations determined by the AI.

In some embodiments, the image data 320 is fused with the diagnostic image data by placing one or more markers corresponding to locations of a lesion as described herein. In some embodiments, the location of the bladder neck BN and the location of the VERU are used to combine the intraoperative image 320 with the diagnostic image to generate the fused image 830. In some embodiments, the fused image is generated after the user has input or configured the location of the bladder neck marker 1647 and the location of the verumontanum marker 1649. In some embodiments the lesion is projected onto the image 320 to generate the fused 830 as described herein.

In some embodiments, the 3D perspective view is configured for the user to rotate the 3D treatment profile and 3D image of the tissue to view interaction of the 3D treatment profile and the tissue with a 3D perspective, which can facilitate treatment planning and improve the user's understanding of the interaction of the energy source from treatment probe 450 with the tissue. In some embodiments, the 3D image shown with the 3D treatment profile comprises a fused 3D image as described herein.

While the user interface can be configured in many ways, in some embodiments, the user interface comprises a control bar 1630 to adjust controls and a navigation bar 1650 to allow the user to navigate through the different stages of setup, planning and treatment. In some embodiments, the user interface 700 comprises a plurality of inputs 630 that allow the user to adjust the treatment profile.

In some embodiments, the control bar 1630 comprises a plurality of controls that allow the user to toggle the image shown on the display and location. For example, control 1632 can be configured for a user to toggle between transverse views and longitudinal images. Additional controls such as illumination, position of the treatment probe and rulers can also be provided.

In some embodiments, the user interface 700 comprises a plurality of user selectable inputs 1640 that allow the user to select among different transverse planes and corresponding locations along the longitudinal image, such as treatment start (TS), location 1, location 2, location 3, location 4, location 5 and treatment end TE. When a location is selected, the directional pad 1680 can be used to move the treatment profile marker. For example, input 1642 corresponding to marker 1644 overlaid on the image, e.g. marker 4, can be selected, and the directional pad used to move the location of treatment profile marker 1644. The movement of the marker can be seen on the longitudinal image of the 3D treatment profile and on the 3D perspective view 1600 in real time, e.g. with latencies less an about one second. Alternatively or in combination, the display may comprise a touch screen display that allows the user to move the treatment profile markers directly on the image by touching the display at the location of the treatment profile marker 1644 overlaid on the image.

In some embodiments, a bladder neck BN is identified with a marker 1647, and a veru protection zone (VPZ) is identified with a marker 1649 to show the user the approximate location of the bladder neck and verumontanum and the corresponding transverse images, which can be helpful for treatment planning around these delicate tissue structures.

In some embodiments, the protection zone such as VPZ is designated by the user such as a surgeon. Alternative or in combination, the VPZ can be automatically created by the system based on detecting the appropriate objects with an AI algorithm as described herein. In some embodiments, the protected zone is identified to protect the delicate tissue structure, such as the verumontanum, by creating a no-treat buffer zone with a buffer angle around the identified delicate tissue structures such as the verumontanum.

In some embodiments, a first ruler 1610 is shown extending in a longitudinal direction near the treatment probe 450, and a second ruler 1620 is shown extending radially from the treatment probe 450, which can facilitate treatment planning.

To plan the 3D treatment profile, the user may be presented with a plurality of transverse images to generate the 3D treatment profile at each of a plurality of locations, e.g. treatment start TS, location 1, location 2, location 3, location 4, location 5, and treatment end (TE).

FIG. 16B shows user interface 700 on a display 425 with a 3D treatment profile 730 viewed in a transverse view 1670 such as with a transverse image 310 and viewed in a 3D perspective view 1600, in accordance with some embodiments. In some embodiments, the 3D treatment profile 730 comprises a transverse profile shown on a transverse image 310. In some embodiments, the treatment profile is overlaid on the fused image 630.

In some embodiments, a real time transverse image, such as a real time fused transverse image, has a plurality of treatment planning markers overlaid thereon. In some embodiments, the real time transverse image 310 comprises a fused image 830, in which the pre-operative diagnostic image is fused with a real time ultrasound image as described herein.

In some embodiments, a real time transverse image 310 has a plurality of treatment planning markers overlaid thereon. In some embodiments, the plurality of treatment planning markers comprises a first marker 1665, a second marker 1666, a third marker 1667 and a fourth marker 1668.

In some embodiments, the user interface comprises a plurality of inputs 620 configured for a user to select each of the plurality of treatment planning markers for adjustment. In some embodiments, a first input 1661 corresponds to a first marker 1665. In some embodiments, a second input 1662 corresponds to a second marker 1666. In some embodiments, a third input 1663 corresponds to a third marker 1667. In some embodiments, a fourth input 1665 corresponds to a four marker 1668. In some embodiments, when a treatment profile marker has been selected with an input, the user interface is configured for the user to adjust the location of the treat profile marker with and input the directional pad 1680, for example by pushing on an arrow of the directional pad to move the marker according to the direction of the arrow.

In some embodiments, the 3D perspective view is configured for the user to rotate the 3D treatment profile and 3D image of the tissue to view interaction of the 3D treatment profile and the tissue with a 3D perspective, which can facilitate treatment planning and improve the user's understanding of the interaction of the energy source from treatment probe 450 with the tissue. In some embodiments, the 3D image of the tissue comprises a fused 3D image of the tissue.

The treatment planning with longitudinal, e.g. sagittal, and transverse images can be performed in any suitable order. For example, the 3D treatment profile can be generated with AI as described herein, or the user can generate the treatment plan from the longitudinal and transverse images, or a combination thereof. In some embodiments, the user may view the 3D treatment plan overlaid over each of a plurality of transverse images followed by viewing the treatment plan overlaid over one or more one or more longitudinal images, e.g. a sagittal images. In some embodiments the user interface is configured for the user to view the treatment profile overlaid over a plurality of transverse images and a plurality of longitudinal images. Allowing the user to view the 3D treatment profile overlaid over a plurality of transverse images and a plurality of longitudinal images can have the advantage of allowing the user to compare the treatment profile with tissue with a plurality of images that intersect each other and allows the user to have a better sense of the treatment profile as compared with the patient's anatomy.

In some embodiments, the real time longitudinal images and the real time transverse images can be acquired more quickly than 3D images, and allowing the user to monitor the treatment in real time with real time longitudinal or real time transverse images (or a combination) can lead to decreased latencies as compared with the user monitoring 3D images. In some embodiments, once the treatment has started, the 3D model is not updated, and the user such as a surgeon relies on a real time longitudinal image such as a sagittal image to view and monitor the treatment in real time. In some embodiments, the intraoperative imaging probe such as a TRUS probe remains fixed during the treatment.

In some embodiments, it will be easier for users such as surgeons to plan a 3D treatment profile on a plurality of different two dimensional (2D) images, in which the 3D treatment profile is overlaid on each of the plurality of 2D images. Work in relation to the present disclosure suggests that this approach may make it easier for surgeons to visualize the treatment profile and associated tissue. As an example, this approach may include defining an angle and depth of treatment on a plurality of transverse images, and defining a length of treatment on a longitudinal image, e.g. a sagittal image or sagittal image slice.

In some embodiments, it may be desirable to maximize the angle of treatment and depth to increase the amount of tissue that can be safely removed. In some embodiments, an automated AI algorithm such as a machine learning (ML) model or neural network is used to identify the boundary of the treatment tissue and determine a treatment angle and depth on the 2D transverse image, e.g. 2D transverse image slice. In some embodiments, the angle and depth of the treatment profile are configured to maximize one or more objectives of the treatment. In some embodiments, the objective of the treatment comprises a maximum amount of target tissue removed in the image such as a transverse image. In some embodiments, the 3D treatment profile shown on the 2D image comprises a cross-section of the 3D treatment at a particular transverse image plane, e.g. a particular point along the longitudinal axis of the imaging probe.

Examples of different treatment objectives include maximizing the treatment area of the 3D treatment profile on the transverse cross-section, and maximizing treatment angle. Other objectives that may apply, such as safety considerations, e.g. avoidance of delicate tissue structures such as the verumontanum and bladder neck.

In some embodiments, the user interface provides automated planning on a transverse slice, and allows the surgeon to review both the anatomical inputs, e.g. tissue boundaries such as the capsule of the prostate, and the proposed treatment removal profile. In some embodiments, the user interface is configured for the user such as the surgeon to choose the desired treatment objective, e.g., maximal area vs. maximal angle vs. maximal resection of an object of interest such as a cancer lesion.

While the treatment planning can be performed with a plurality of user selectable inputs as described herein, in some embodiments the system is configured to suggest one or more transverse or longitudinal image planes to perform this planning in response to identified areas of interest. For example, the system may be configured to identify one or more anatomical planes in response to one or more of an intravesical prostatic protrusion (IPP), a bladder neck (BL), a mid-prostate (MID), or a verumontanum (VERU) and provide these corresponding images on the display for the user. In some embodiments, the system is configured to identify patient-specific areas of interest, for instance based on the imported pre-operative images that may include cancerous lesions at areas and locations of the pre-operative images. By mapping the area of interest such as a lesion onto the 3D model, the system may suggest to the surgeon that treatment planning is desired at these areas.

In some embodiments, the system is configured to automatically generate a 3D treatment plan that nearly fully conforms to a 3D model of the prostate. In some embodiments, the 3D treatment plan follows the boundary of the target tissue, e.g. follows the prostate boundary, and in some instances nearly exactly follows the boundary of the tissue. In some embodiments, the system is configured to create any suitable number of plans for treatment at each of a plurality of images, e.g. at each of a plurality of image slices. In some embodiments, the plurality of images comprises sufficiently high resolution that the treatment plan can be generated for each image plane without requiring interpolation. In some embodiments, the system is configured to generate a treatment plan at each of approximately 50 to approximately 100 planes of approximately 50 to approximately 100 corresponding images. Each slice could be automatically planned by the system to achieve the desired objective, e.g. maximal area or maximal angle, and a stack of these images slices, e.g. image slices, could describe an object to be treated and corresponding treatment plan over a corresponding length, such as a length within a range from about 50 mm to about 100 mm.

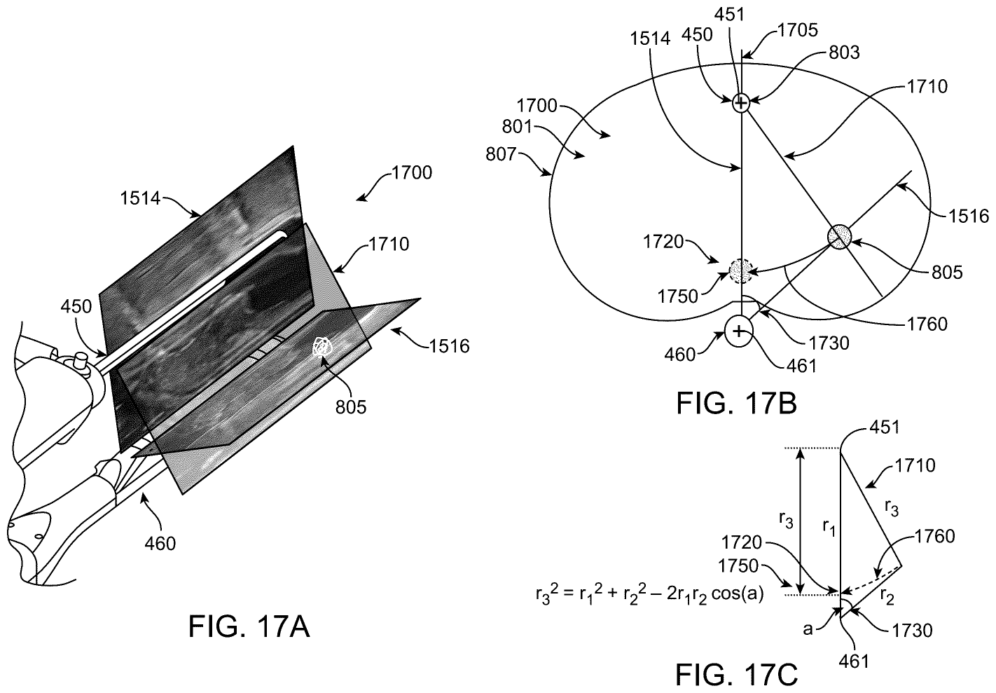

FIG. 17A shows projection of a lesion 805 from a first longitudinal image 1516 to a second longitudinal image 1514 using a treatment probe 450 as a reference, in accordance with some embodiments. In some embodiments, an image 1710 can be generated which extends between the treatment probe and through the lesion 805, for example from the 3D image.

In some embodiments, a frame of reference 1700 is used to project the lesion 805 onto the longitudinal image 1514, which may comprise a sagittal image. In some embodiments the frame of reference 1700 comprises the axis of the treatment probe 450, and the lesion 805 is projected onto the image 1514 by rotating the location of the image about the treatment probe axis onto the longitudinal image 1514.

FIGS. 17B and 17C show dimensions and mapping to project lesion 805 onto an image that is visible to the user and shown on the user interface as a ghost lesion, in accordance with embodiments. In some embodiments, the projected lesion is shown as a ghost image because the lesion is not physically located in the plane of the image, although the lesion is located proximally to the plane of the image. In some embodiments, the presence of the ghost lesion in an image allows the user to adjust the treatment profile, e.g. the 3D treatment profile. In some embodiments, this allows the user to treat tissue away from the physical location of the lesion. This treatment of tissue away from the lesion in addition to treatment of the lesion itself can help to provide a wide margin of treatment around the lesion and complete removal of tissue associated with the lesion, for example.

FIG. 17B shows an axial view of three planes as in FIG. 17A viewed from a corresponding transverse image. The lesion 805 is shown on longitudinal image 1516 at a distance from imaging probe 460, e.g. at a distance from imaging probe axis 461. The projected lesion 1750 is shown at a location on image 1514. In some embodiments, the location 1720 of the projected lesion 1750, e.g. the ghost lesion, corresponds to the distance from the treatment probe 450 to the lesion 805, for example by rotating the location of the lesion 805 about the treatment probe axis 451 as indicated with arc 1760. This projection of the lesion 805 maintains the radial distance from the treatment probe to the lesion 805 with a similar radial distance from the treatment probe to the projected lesion 1750. Maintaining the radial distance can be helpful with treatment planning, because the user can adjust the treatment profile in the longitudinal image, e.g. sagittal image, so as to treat the lesion.

In some embodiments, the lesion 805 is shown at a location along image 1710 at a distance from the treatment probe 450.

FIG. 17C shows radii and corresponding angles and the calculation of a projection of the lesion from the first longitudinal image to the second longitudinal image, in accordance with the embodiments of FIGS. 17A and 17B. In some embodiments, the treatment probe is separated from the imaging probe by a distance r₁. The lesion 805 is present in image 1516, which may comprise a longitudinal ultrasound image or a fused longitudinal image as described herein. In some embodiments, the longitudinal image 1516 with the lesion 805 is at an angle 1730 (angle α) with respect to the longitudinal image 1514 upon which the lesion is to be projected. The lesion 805 is located in image 1516 at a distance r₂ from the imaging probe 460, for example at a distance from the imaging probe axis 461. The distance r₃ is the distance from the treatment probe 450 to the location of the projected lesion 1750 shown as a ghost lesion on image 1514.

In some embodiments, the distance from the treatment probe to the projected lesion shown on the display, e.g. the ghost lesion, is determined based at least in part of the formula:

(r₃)²=(r₁)²+(r₂)²−2r₁r₂ cos(a).

One or more of the longitudinal images 1514, 1516 and 1710 can be show in the user interface on the display with the 3D treatment profile overlaid thereon as described herein, for example in accordance with FIG. 16A.

In some embodiments, the 3D treatment profile is overlaid on the transverse image shown in FIG. 17B, which may comprise an ultrasound image or a fused transverse image (and combinations thereof), and the user is able to provide an input to the user interface to provide a view the 3D treatment profile overlaid on the images shown in FIG. 17B, such as the treatment profile overlaid on one or more of image 1514, image 1516 or image 1710. The transverse view shown in FIG. 17B may comprise additional images as appropriate, for example image 1512 as shown in with reference to FIG. 15A

Although FIGS. 17A to 17C refer to projecting a lesion onto an image with reference to rotation of the lesion location about a treatment probe axis, other approaches may be used. For example, other approaches can be used such as coordinate references such as cartesian coordinate references, or vector projections, and combinations thereof. In some embodiments, the 3D spatial coordinates of the lesion 805 are mapped onto the image such as image 1514 in response to the 3D spatial coordinates of the lesion and the 3D spatial coordinates of the image. Alternatively or in combination, a vector projection of the lesion 805 from image 1516 onto image 1514 may be used. In some embodiments, the vector projection mapping of the lesion 805 from the image 1516 to the image 1514 changes the distance from the treatment probe to the projected lesion, and the user interface is configured for the user to plan the treatment accordingly, for example with additional images such as transverse images.

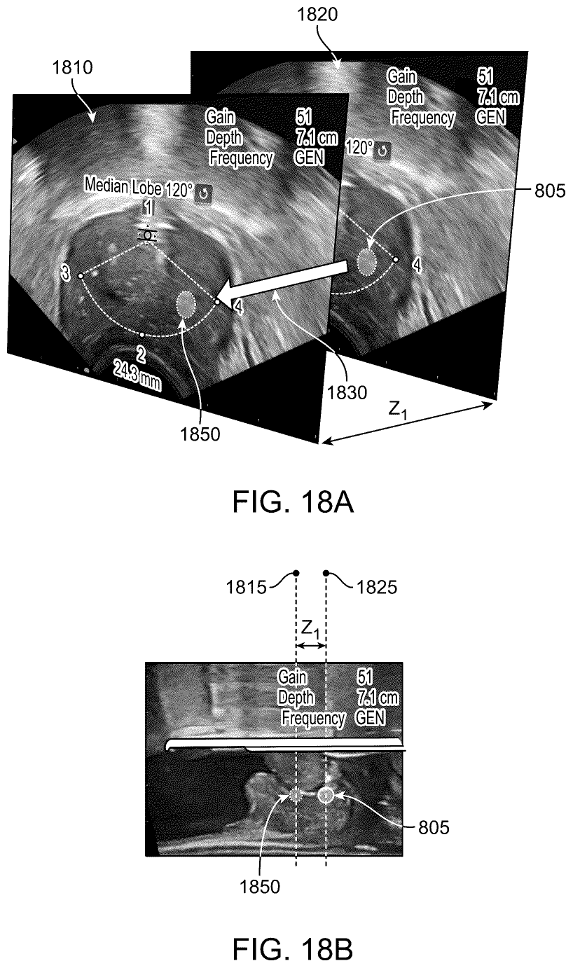

FIG. 18A shows two transverse images and projection of a lesion 805 from a first image 1820 to a second image 1810, in accordance with some embodiments. In some embodiments, the lesion 805 is located in a first image 1820, which is separated from second image 1810 by a distance Z₁. The lesion 805 on image 1820 is projected onto image 1810 and shown as projected lesion 1850. The projected lesion 1850 can be shown in any suitable way on image 1810 and may comprises a ghost lesion with an appropriate indicium to indicate the projected lesion 1850 is located away from image 1810. This approach can inform the user of proximity to the image 1810 to the lesion, so that the user can adjust the treatment plan accordingly. The transverse images 1810 and 1820 can be show in the user interface on the display with the 3D treatment profile overlaid thereon as described herein, for example in accordance with FIG. 16B.

FIG. 18B shows locations of the transverse images of FIG. 18A on a longitudinal image such as a sagittal image, in accordance with some embodiments. The image 1810 extends along a plane 1815 and the image 1820 extends along a plane 1825. The plane 1815 is separated from the plane 1825 by the distance Z₁. The lesion 805 on plane 1825 of image 1820 is projected as lesion 1850 onto image 1810 on plane 1815. The projected lesion 1850 may appear in transverse image 1810 as a ghost lesion as described herein. In some embodiments, the spatial coordinates and location of the lesion 1850 in transverse image 1810 correspond with the spatial coordinates and location of the lesion 805 in image 1820.

In some embodiments the lesion 805 and the projected lesion 1850 as shown in FIG. 18B appear on one or more of the longitudinal images with the 3D treatment profile overlaid thereon as described herein.

The projected lesions as described herein, e.g. ghost lesions, may comprise any suitable indicium to indicate that the lesion comprises a projected lesion, such as one or more of highlighting, shading, transparency, size, patterns or other visual cues to indicate the projective-nature of the representation shown on the display.

In some embodiments, the projection and associated indicium is related to the type of energy source as described herein. Also, the approach used to project the lesion from one location to another can vary depending on the type of energy source used for treatment.

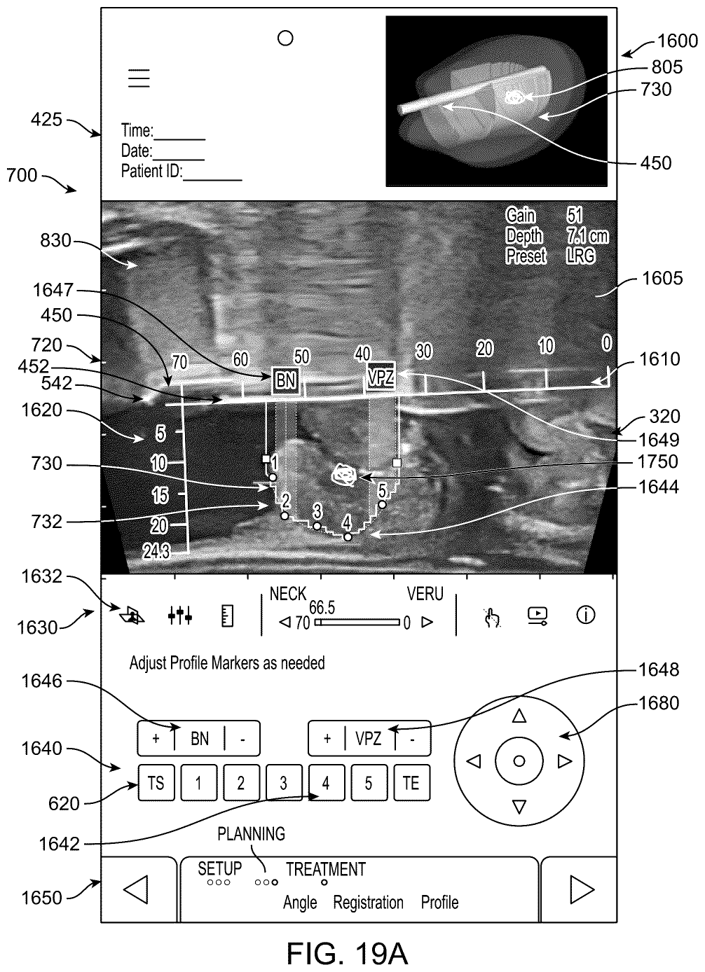

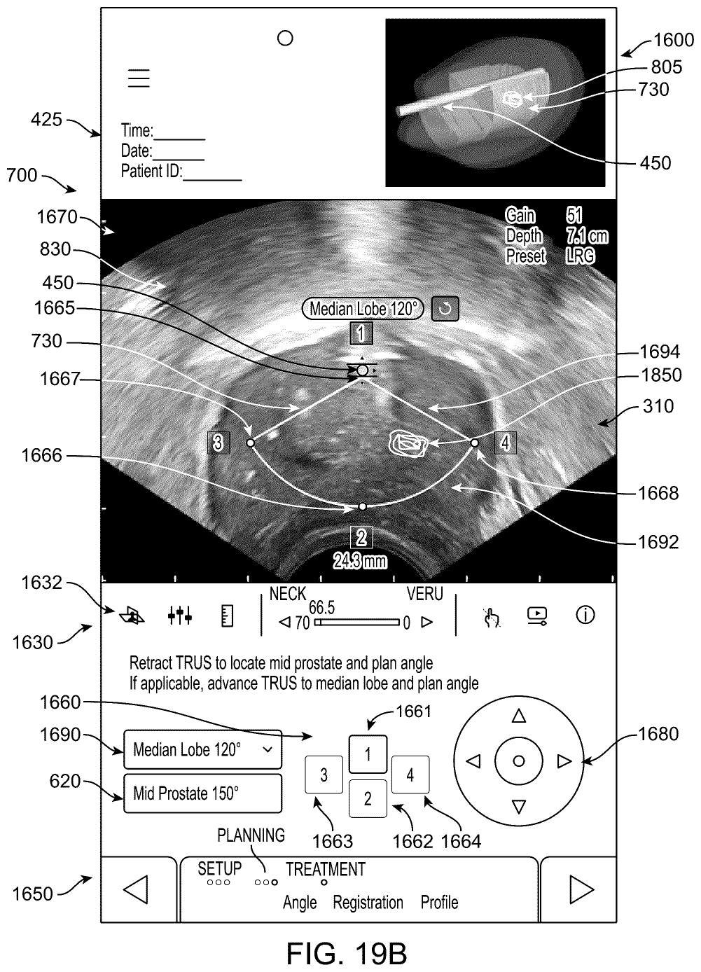

FIG. 19A shows a projected lesion 1750, e.g. a ghost lesion, projected onto a longitudinal image such as a sagittal image for treatment planning, for example as described herein with reference to FIG. 16A. In addition to the longitudinal image, the 3D treatment profile is shown overlaid on the longitudinal image, such a sagittal image, which allows the user to visualize the treatment with a 3D perspective view 1600. The 3D image is shown with the lesion 805 and the 3D treatment profile overlaid thereon with the 3D perspective view 1600.

FIG. 19B shows a projected lesion 1850, e.g. a ghost lesion, projected onto a transverse image for treatment planning, for example as described herein with reference to FIG. 16B. In addition to the transverse image, the 3D treatment profile is shown overlaid on the transverse image, which allows the user to visualize the treatment with a 3D perspective view 1600. The 3D image is shown with the lesion 805 and the 3D treatment profile overlaid thereon with the 3D perspective view 1600.

In some embodiments, providing the 3D perspective view on the display with one or more of the longitudinal image or the transverse image has the advantage of allowing the user to better visualize the location of the lesion 805 and one or more of the projected lesion 1750 or the projected lesion 1850 shown on the corresponding longitudinal or transverse image.

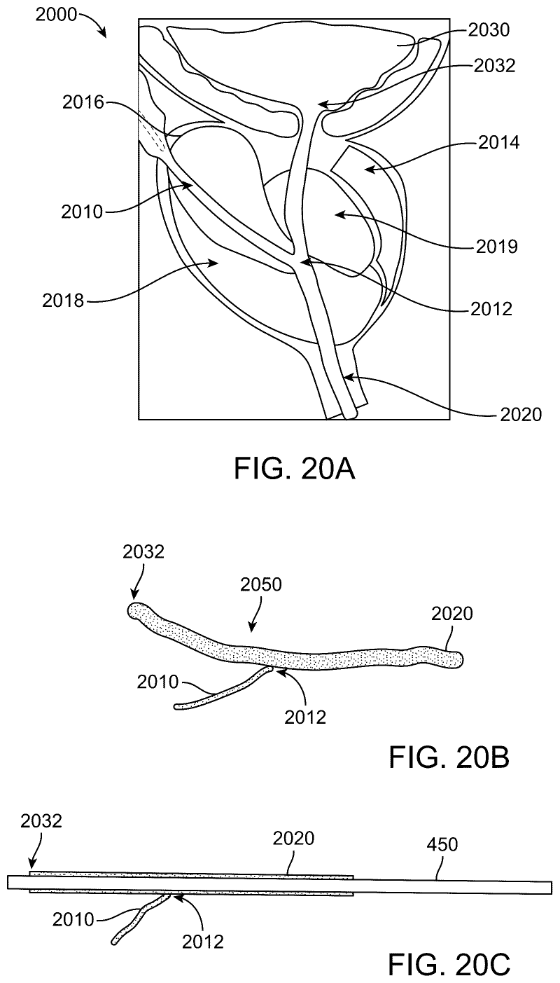

FIG. 20A shows a lumen such as a prostate 2000 and associate tissue structures, in accordance with some embodiments. A seminal vesical 2010 extends to a verumontanum 2012 of the prostate 2000. The prostate may include additional tissue such as the anterior fibromuscular stroma 2014, a central zone 2016 a peripheral zone 2018, and a transition zone 2019, for example. A urethra 2020 extends from a distal tip of the penis, through the prostate 2000, and into a bladder 2030. The urethra 2020 is coupled to the bladder 2030 at a bladder neck 2032. The bladder neck 2032 comprises a group of muscles that connect the bladder to the urethra and tighten to hold urine in the bladder, and relax to release urine through the urethra.

FIG. 20B shows a lumen such as a urethra of a prostate and other anatomical structures prior to insertion of a probe. Prior to insertion of the probe into the prostate, the urethra extends through the prostate with a curved path 2050. In some embodiments, the urethra 2020 is curved between the verumontanum 2012 and the bladder neck 2032.

FIG. 20C shows a lumen such as a urethra of a prostate with a probe 450 inserted into the urethra 2020. In some embodiments, the probe comprises a stiffness greater than the urethra and the urethra comprises a shape that corresponds to the shape of the probe. While the probe can be shaped in many ways, in some embodiments, the probe comprises a substantially straight profile so as to straighten the urethra 2020 between the verumontanum and the bladder neck. In some embodiments, the substantially straight probe comprises an amount of deflection of no more than about 10 degrees in a free standing configuration.

In some embodiments, shape profile data from the probe is used to combine image data from the diagnostic image with the intraoperative image. In some embodiments, the shape profile data of the probe is used to generate the fused images as described herein. In some embodiments, and the diagnostic image data is projected onto the intraoperative images in response to the shape profile data of the probe. For example, in the diagnostic image the urethra may comprises a curved shape profile, In some embodiments, the probe comprises a substantially straight probe, and the urethra is straightened with the probe placed therein. Although reference is made the to the urethra, this approach be used with any lumen of any tissue of any organ as described herein. Although reference is made to a straight probe, the probe may comprise any suitable shape such as a curved shape profile.

In some embodiments, the diagnostic image comprises data that is not readily apparent in an intraoperative image such as an ultrasound image. In some embodiments, data from additional intraoperative images can be used to determine the location of one or more tissue structures. For example, the verumontanum may not be readily identified in an intraoperative ultrasound image such as a longitudinal image, e.g. sagittal image, from a TRUS probe. In some embodiments, an endoscope such as a cystoscope is inserted with the treatment probe, and the location of the endoscope is visible in the ultrasound image. When the endoscope is near the verumontanum, the verumontanum is visible in the endoscope image and the endoscope is visible in the intraoperative image. For example, the verumontanum may be within about 5 mm to about 10 mm from the tip of the endoscope. In some embodiments, the position of the endoscope in the ultrasound image is used to determine the position of the verumontanum. In some embodiments, the user interface is configured for the user to provide an input when the verumontanum is visible in the endoscope image. Alternatively or in combination, the AI can be configured to identify the verumontanum in the endoscope image. The location of the endoscope in the intraoperative image can be used to determine the location of the verumontanum in the intraoperative image, in response to the verumontanum being within the field of view of the endoscope.

In some embodiments, elements that are hidden and/or deformed after the treatment probe is inserted (e.g., hidden from ultrasound by the hyperechoic shadow created by the treatment probe) can be correlated between the two 3D objects and allow items previously visible in the pre-insertion diagnostic image to be mapped to appropriate locations on the intraoperative image, which may comprise a deformed 3D model of the tissue.

In some embodiments, the model of the organ in undeformed configuration is used to fuse the diagnostic image with the intraoperative image. Alternatively or in combination, the deformed 3D model of the tissue can be used in fusing the diagnostic image with the intraoperative image. In some embodiments, locations of the markers such as the bladder neck and verumontanum from the deformed model are used to map other tissue structures such as the lesion shape and lesion location from the deformed model to the intraoperative image. In some embodiments, the lesion is present in the model and deformed in response to the probe shape. The deformed lesion data is then mapped onto the intraoperative image to generate the fused image.

In some embodiments, pre-operative imaging such as an MRI will be available that may contain areas of interest, such as one or more lesions. This pre-operative imaging may be of better or different image quality (e.g., resolution, contrast) that allows different visibility of items than may be available with intraoperation imaging. In some embodiments, those pre-operative diagnostic images can be imported into the system e.g., by connection to a PACS (Picture Archiving and Communication System). Alternatively or in combination, pre-operative diagnostic data can be imported via external memory stick or a network connection, for example. The pre-operative diagnostic images can then be fused to the 3D models in accordance with known image fusion techniques as will be understood by one of ordinary skill in the art.

In some embodiments, common points, e.g., fiducials, are registered between the diagnostic image and the intra-operative image. The fiducials may comprise tissue of one or more tissue markers as described herein, such as the verumontanum and bladder neck, and may comprise artificial markers, for example.

The image fusion can be performed in many ways and may comprise one or more of rigid fusion or elastic fusion, and combinations thereof, for example.

The image fusion may be accomplished through a plurality of steps. Examples of steps include 1) pre-operative imaging, 2) imaging with an ultrasound inserted into the patient and prior to insertion of the treatment probe and 3) imaging with the ultrasound probe and the treatment probe inserted into the patient, and imaging In some embodiments, an initial fusion to the initial scan of the imaging probe (before insertion of the treatment probe) may be easier to perform and/or can be performed with higher confidence, and follow with a second mapping from the fused objects to the post-insertion 3D model. The multi-step fusion may allow more accuracy than a single step fusion.

In some embodiments, a separate 3D model may be created pre-operatively, in which the 3D model may contain areas of interest as described herein. In some embodiments, two 3D models may be mapped together. In some embodiments, the accuracy of the mapping may be improved with a physics-based deformation model, in which deformation is applied to the original model prior to mapping, for example. This deformation may be performed using known physics techniques, a machine-learning based approach or a combination of thereof, for example.

In some embodiments, areas of interest in pre-operative images and objects are mapped to the 3D model. The 3D model can be generated from intraoperative images or preoperative diagnostic images and combinations thereof. In some embodiments, the 3D model is mapped to the intraoperative images. Alternatively or in combination, the intraoperative images are mapped to the 3D model, and the 3D model comprises the fused image data.

In some embodiments, the areas of interest are identified through automated techniques such as processing the images with an AI algorithm as described herein, such as a neural network or machine learning algorithm. In some embodiments, the intraoperative images received from the real time intraoperative images are used to identify objects. In some embodiments, this includes identifying the treatment probe, specific parts of the treatment probe, and tissue structures as described herein, such as one or more of a lesion, a lumen, a urethra, a capsule, a verumontanum or a bladder neck, for example. In some embodiments, the tissue structure comprises one or more cancerous lesions.

In some embodiments, additional live imaging modalities may be available in addition to the primary imaging modality that created the initial scans for the 3D models. In some embodiments, a TRUS imaging probe is rotated longitudinally to acquire a plurality of longitudinal images as described herein and create a 3D model of the prostate. In some embodiments, the treatment probe comprise an endoscope such as a cystoscope. In some embodiments, an endoscopic view, e.g. a cystoscopic view is available as the treatment probe is inserted, and there are both live ultrasound images from the TRUS probe as well as live cystoscopic images of the urethra. In some embodiments, items of interest (e.g. markers) may be identified from the endoscopic view, e.g. cystoscopic view, such as the external sphincter, the verumontanum or the bladder neck, for example. As the treatment probe and endoscope (e.g. cystoscope) can be seen in a longitudinal (e.g. sagittal) ultrasound view created by the TRUS imaging probe, the location of items viewed by the endoscope (e.g. cystoscope) can be mapped to a location in the ultrasound image and the 3D model. In some embodiments, the user interface is configured for the user to input the location of the items of interest on the ultrasound images and fused images by moving one or more markers on the user interface as described herein.

In some embodiments, the areas of interest are pre-identified (e.g., labeled in pre-op images), automatically identified by the system in the interoperative images with an AI algorithm, or identified by the surgeon using real time visualization, and combinations thereof, as described herein.

In some embodiments, the user interface is configured to allow the user such as a surgeon to identify areas of interest. In some embodiments, the user interface is configured for the surgeon to accept, deny or modify (e.g., move location and/or size) the identified area of interest, e.g. markers, as described herein.

In some embodiments, the fused images are constructed from the model of the tissue, such as a model of an organ, for example a model of the prostate.

Once the 3D model has been generated, it is possible to generate/reconstruct any desired 2D slice in any plane from the model, for example with a Digitally Reconstructed Radiograph (“DRR”). In some embodiments, 2D transverse images slices are generated in the treatment probe plane and orthogonal to the treatment probe travel direction. This can allow effective and appropriate planning of treatment with the energy released from the treatment probe as described herein. In some embodiments, 2D longitudinal images aligned through the treatment probe are constructed. In some embodiments, 2D longitudinal image at 12 o'clock comprises a sagittal plane image.

In some embodiments, during planning of the treatment, it may be helpful to visualize both the 3D treatment plan and the relative position inside a 3D anatomical model. For instance, a resection of the prostate may include a treatment probe's planned 3D resection profile inside a 3D model of the prostate.

In some embodiments, it is helpful if the model of the treatment and of the anatomical tissue structures can be rotated and zoomed such that the user can see the model from any angle.

In some embodiments, where planning is performed on a 2D image, e.g. a 2D image slice, both the 3D model and the 2D image with the treatment profile overlaid thereon may be shown at the same time, such that changes to the 2D model show in real-time as changes to the 3D resection profile.

In some embodiments, a model of the tissue of an organ such as the prostate is generated from the diagnostic image or from a 3D image obtained with a probe inserted into the patient. While the model can be configured in many ways, in some embodiments, the model comprises 3D finite element model. In some embodiments, the diagnostic image is segmented into different tissue types with a trained AI algorithm as described herein, and the finite element model generated from the segmented 3D image.

In some embodiments, the 3D model is constructed with a commercially available tool, such as an open source tool, for example, Blender, as will be known by one of ordinary skill in the art. In some embodiments, the model is constructed with one or more of the diagnostic image or the intraoperative image, or a combination thereof.

In some embodiments, sequential 2D image slices are acquired at regular intervals. In some embodiments such as with transverse prostate slices, the slices are acquired every mm along a z-axis through the urethra. In some embodiments for longitudinal image slices such as sagittal image slices, the spacing can be every 0.5 degrees, or other appropriate interval. In some embodiments, the spacing is configured to provide coverage based on the depth of interest along a rotational axis of the TRUS probe in the rectum. Alternatively or in combination, the model can be generated from the diagnostic image.

In some embodiments, the image slices are stacked to form a 3D grid of voxels.

In some embodiments, one or more surfaces are constructed (also referred to as reconstruction) with a mesh grid such as a triangular mesh grid.

In some embodiments, a model of tissue such as tissue of an organ comprises a virtual object of the tissue and may comprise a virtual object of an organ. In some embodiments, the virtual object is used to generate the fused images as described herein.

In some embodiments, post-processing of the one or more surfaces is performed for any suitable cleanup, for example to remove roughness from the surface. While any suitable surface can be modeled, in some embodiments, the surface comprises an internal surface of the urethra. Examples of surfaces that can be modeled in include one or more of the urethra, the capsule of the prostate, and the surface of the bladder neck, the surface of the seminal vesicles, the surface of the verumontanum, or the intersection of surfaces. In some embodiments, the intersection of internal surfaces of one or more vessels is modeled, such as the intersection of the internal wall seminal vesicles and the internal wall of the urethra near the verumontanum.

In some embodiments, the 3D model, e.g. the virtual object, is deformed using one or more of forces or displacement. In some embodiments, the tissue is shaped with the probe, for example. While the deformation can be modeled in many ways, in some embodiments the 3D model is deformed using forces, such as with a feed forward or open loop deformation process as will be understood by one of ordinary skill in the art of computer modeling such as finite element modeling.

In some embodiments, the modeled 3D virtual object is represented by one or more of a surface mesh, voxel grid or other representation.

In some embodiments, material properties are assigned to each type of tissue. Example material properties include elasticity and density.

In some embodiments, a physics engine models the forces and deformations. In some embodiments, the physics engine comprises a Finite Element Model.

In some embodiments, an updated mesh is calculated and can be visualized.

In some embodiments, an open loop deformation can be assisted by constraints of known anatomical features and positions. Examples of such constraints include the shape of the probe as described herein.

In some embodiments, the same object is modeled in different configurations. For example, the prostate can be modeled as a virtual object without a probe inserted into the patient and with one or more probes inserted into the patient. In some embodiments, the prostate is modeled as a virtual object with one or more of a TRUS probe inserted into the patient or a treatment probe inserted into the urethra, and combinations thereof.

In some embodiments, the modeled shape of the organ such as the prostate differs in response to the placement of one or more probes, or in response to a different type of imaging modality, such as ultrasound and MRI, for example.

In some embodiments, an alignment is performed. The alignment may comprise one or more of a global registration, or local fiducials that have identified, such as for registration purposes or treatment planning purposes, as described herein.

In some embodiments, correspondence is established with one or more of feature matching or manual annotation, and combinations thereof, for example with inputs to the treatment planning user interface as described herein.

In some embodiments, a deformation mapping is determined, e.g. calculated. In some embodiments, this deformation mapping comprises a transformation vector that is applied to the 3D model, e.g. applied to the 3D virtual object.

In some embodiments, warping is performed to transfer geometry and other data between models.

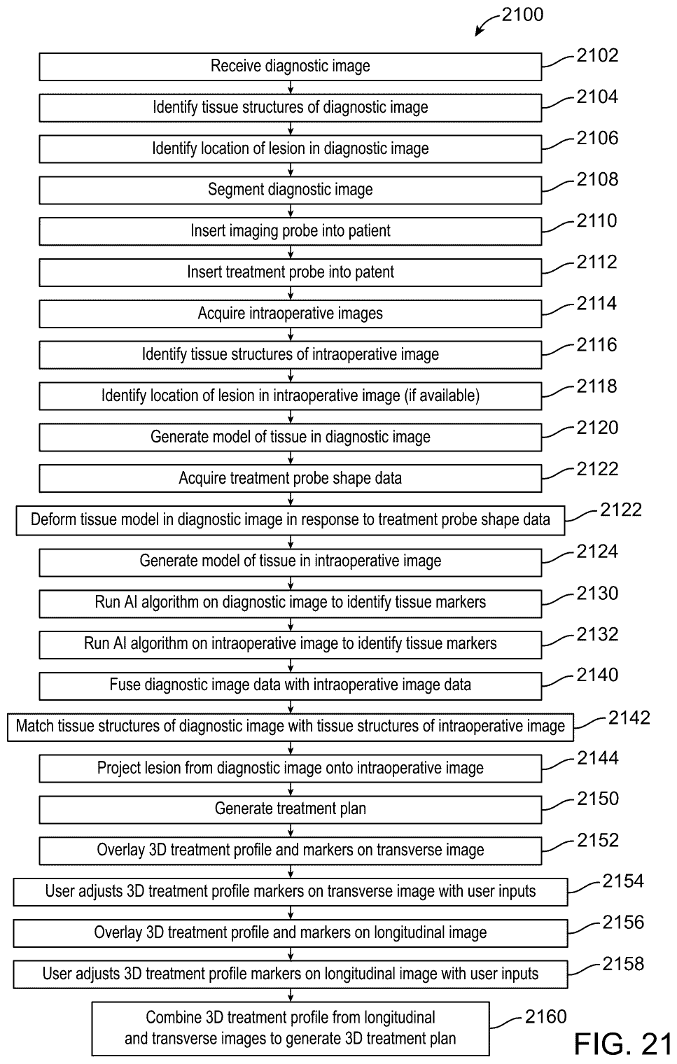

FIG. 21 shows a method 2100 of generating a treatment plan, in accordance with some embodiments.

At a step 2102, diagnostic image is received. The diagnostic image data may comprise any suitable diagnostic image data as described herein, such as MRI image data. In some embodiments, the diagnostic image data comprises 3D image data, e.g. 3D MRI data.

At a step 2104, tissue structures of diagnostic image are identified. The tissue structures can be identified with an AI algorithm, or a user inputting the locations on a user interface, and combinations thereof, as described herein.

At a step 2106, a location of lesion is determined in the diagnostic image. The location can be determined with an AI algorithm or input by a user interface, or combinations thereof, as described herein.

In some embodiments, a profile of the lesion is determined. The profile of the lesion can be determined with an AI algorithm or user input, and combinations thereof. In some embodiments, the profile of the lesion from the diagnostic image is projected onto the intraoperative image to generate the fused image.

At a step 2108, the diagnostic image is segmented into different tissue types. The diagnostic image data can be segmented into different tissue types in accordance with the imaged tissue. In some embodiments, the tissue is segmented into one or more of capsular tissue, glandular tissue, lumen tissue, internal lumens, lumen walls, vesicles, stromal tissue, tumor tissue, urethral tissue, bladder neck tissue, or verumontanum tissue, for example.

At a step 2110, an imaging probe is inserted into the patient. The imaging probe may comprise any suitable probe, such as an ultrasound probe. In some embodiments, the imaging probe comprises a TRUS probe.

At a step 2112, a treatment probe is inserted into the patent. The treatment probe may comprise any suitable probe as described herein.

At a step 2114, intraoperative images are acquired. In some embodiments, the intraoperative images are acquired to generate a 3D intraoperative image. The 3D intraoperative image may comprise a 3D ultrasound image, for example. In some embodiments, the 3D ultrasound image is generated by rotating the imaging probe to a plurality of rotational acquisition angles as described herein. In some embodiments, the longitudinal position of the ultrasound probe remains fixed while the ultrasound probe is rotating to the plurality of rotational angles. Alternatively or in combination, the 3D ultrasound image can be generated by translating the ultrasound probe to a plurality of longitudinal locations, for example by advancing the ultrasound probe to a plurality of locations or retracting the ultrasound probe to a plurality of locations. In some embodiments, the rotational angle of the ultrasound probe remains fixed while the ultrasound probe is translated to the plurality of longitudinal locations.

In some embodiments, the imaging probe is used to acquire a plurality of 2D intraoperative images to generate a 3D image for treatment planning, and to subsequently generate a real time 2D image, such as a real time longitudinal image, for treatment monitoring. In some embodiments, the real time longitudinal image comprises a real time sagittal image from a TRUS probe.

At a step 2116, tissue structures are identified in the intraoperative image. The tissue structures can be identified by an AI algorithm, or a user, and combinations thereof.

At a step 2118, a location of the lesion is identified in the intraoperative image (if available). The lesion can be identified by an AI algorithm, or a user input, and combinations thereof.

At a step 2120, a model such as a virtual object is generated of tissue in diagnostic image.

At a step 2122, treatment probe shape data is acquired. The treatment probe may comprise any suitable shape such as straight or curved for example.

At a step 2122, the tissue model such as the virtual object is deformed in response to treatment probe shape data. In some embodiments, the treatment probe shape is used to deform the virtual object, for example by providing constraints to the model to conform the virtual object to the shape of the treatment probe.

At a step 2124, a model of tissue in in the intraoperative image is generated, for example by generating a virtual object comprising the tissue.

At a step 2130, an AI algorithm processes the diagnostic image to identify tissue markers. The diagnostic image may comprise a 3D diagnostic image, for example. Alternatively or in combination, the diagnostic image may comprise a plurality of 2D images.

At a step 2132—an AI algorithm processes the intraoperative image to identify tissue markers. The intraoperative image may comprise any suitable intraoperative image as described herein, such as a 3D intraoperative image. Alternatively or in combination, the intraoperative image may comprise a plurality of 2D images.

At a step 2140, diagnostic image data is fused with intraoperative image data.

At a step 2142, tissue structures of the diagnostic image are matched with tissue structures of intraoperative image.

At a step 2144, the lesion data from diagnostic image data is projected onto intraoperative image data.

At a step 2150, a treatment plan is generated.

At a step 2152, a 3D treatment profile and markers are overlaid on transverse images.

At a step 2154, a user adjusts the 3D treatment profile markers on transverse image with user inputs.

At a step 2156, a 3D treatment profile and markers are overlaid on one or more longitudinal images.

At a step 2158, a user adjusts the 3D treatment profile markers on longitudinal image with user inputs.

At a step 2160, 3D treatment profile from longitudinal is combined with transverse images to generate 3D treatment plan.

Although FIG. 21 shows a method 2100 of generating a treatment plan in accordance with some embodiments, one of ordinary skill in the art will recognize many adaptations and variations in accordance with the present disclosure. The method 2100 may comprise additional or alternative step of any method as described herein. For example, some of the steps can be removed, some of the steps repeated, some of the steps may comprise sub-steps of other steps, and the steps can be performed in any order.

Although the steps of the method 2100 can be performed in any suitable order, in some embodiments, one or more of the steps are performed sequentially. In some embodiments, acquiring a plurality of 2D intraoperative images to generate a 3D intraoperative image is performed prior to fusing 3D diagnostic image data with the 3D intraoperative image to generate a 3D fused image, which are performed prior to generating a 3D treatment plan in response to the 3D fused image. In some embodiments, a real time 2D image is provided on a display for a user to monitor treatment after the 3D treatment plan is generated in response to the fused image.

In some embodiments the method 2100 comprises a method of generating a three dimensional (3D) treatment plan. In some embodiments, the method comprising: receiving 3D diagnostic image data; generating a 3D intraoperative image; fusing 3D diagnostic image data with 3D intraoperative image data to generate a fused 3D image; generating a plurality of fused two dimensional (2D) images from the fused 3D image; generating a 2D treatment plan in response to data from each of the plurality of fused 2D images; and generating a 3D treatment plan in response to the plurality of 2D treatment plans.

In some embodiments, each of the plurality of fused 2D images is presented to the user on a display with a plurality of markers overlaid thereon and the user interface is configured for the user to adjust the plurality of markers and the treatment profile displayed on said each of the plurality of fused images.

In some embodiments, the plurality of fused 2D images comprise Digitally Reconstructed Radiographs generated from the fused 3D image.

In some embodiments, the plurality of fused images comprises a plurality of fused transverse images generated from the fused 3D image.

In some embodiments a real time 2D image is provided on a display for a user to monitor treatment.

In some embodiments the real time 2D image comprise a real time fused 2D image and optionally wherein the real time fused 2D image comprises a real time sagittal image with the lesion projected thereon with a plurality of markers.

In some embodiments, the plurality of fused 2D images is processed with an AI algorithm to generate the 2D treatment plan on said each of the plurality of fused 2D images.

In some embodiments, the user interface is configured to display the AI generated 2D treatment plan on said each of the plurality of fused 2D images and configured to receive inputs for the user to adjust the AI generated 2D treatment plan and corresponding markers on said each of the plurality of fused 2D images.

In some embodiments, the user interface is configured to display the 2D treatment plan on said each of the plurality of fused 2D images and configured to receive inputs for the user to adjust the 2D treatment plan and corresponding markers on said each of the plurality of fused 2D images.

The processor can be configured to perform any of the steps of method 2100.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

As used herein, the terms first, second, third, etc. are merely meant to be descriptive without reference to any particular order, and can be used interchangeably, unless indicated otherwise.

The present disclosure includes the following numbered clauses.

Clause 1. A method of treating tissue of a patient, the method comprising: arranging a treatment probe and an imaging probe to have a first offset with respect to a midline of the patient in a first configuration; treating a first region of tissue with an energy source from the treatment probe in the first configuration; arranging the treatment probe and the imaging probe to have a second offset with respect to the midline of the patient in a second configuration; treating a second region of the tissue with the energy source from the treatment probe in the second configuration.

Clause 2. The method of clause 1, wherein the treatment probe comprises a handpiece.

Clause 3. The method of any of the preceding clauses, further comprising rotating a handpiece around a longitudinal axis of the treatment probe.

Clause 4. The method of any of the preceding clauses, wherein the handpiece comprises a first orientation in the first configuration and a second orientation in the second configuration, the second orientation opposite the first orientation.

Clause 5. The method of any of the preceding clauses, wherein the handpiece is rotated by an amount within a range from about 90 degrees to 270 degrees.

Clause 6. The method of any of the preceding clauses, wherein the energy source is located on a second side of the patient with respect to the midline to treat the first region, the first region on a first side of the patient and wherein the energy source is located on the first side of the patient to treat the second region.

Clause 7. An apparatus to treat tissue of a patient, the apparatus comprising: a treatment probe comprising an energy source and a handpiece; an imaging probe configured to image the tissue; and a processor operatively coupled to the treatment probe and the imaging probe, the processor configured to display a fused image on a display with a treatment plan overlaid on the fused image, the processor configured to receive user inputs to adjust the treatment plan overlaid on the fused image.

Clause 8. The apparatus of clause 7, wherein the handpiece is configured to rotate between a first orientation to treat tissue on a first side of the patient and a second orientation to treat tissue on a second side of the patient.

Clause 9. The apparatus of any of the preceding clauses, further comprising a first lockable arm configured to support the treatment probe with a first offset with respect to a midline of the patient in a first configuration and support treatment probe with a second offset with respect to the midline of the patient in a second configuration, wherein the treatment probe is configured to treat a first region of tissue with the energy source from the treatment probe in the first configuration and to treat a second region of the tissue with the energy source from the treatment probe in the second configuration.

Clause 10. The apparatus of any of the preceding clauses, the energy source and the handpiece comprise a first orientation in the first configuration and a second orientation in the second configuration, the second orientation opposite the first orientation.

Clause 11. A method of generating a treatment plan, the method comprising: receiving a diagnostic image of a patient, the diagnostic image comprising one or more lesions; receiving an intraoperative image of the patient; and combining data from the diagnostic image with data from the intraoperative image to generate a fused image with the lesion projected onto the fused image.

Clause 12. The method of clause 11, further comprising: determining a location of a treatment probe and projecting the lesion onto the fused image in response to the location of the treatment probe.

Clause 13. The method of any of clauses 11 to 12, wherein the lesion is located a radial distance from the treatment probe and the lesion is projected onto the fused image at the radial distance from the treatment probe to the lesion.

Clause 14. The method of any of clauses 11 to 13, wherein the intraoperative image extends along a plane and the lesion is located away from the plane.

Clause 15. The method of any of clauses 11 to 14, wherein the treatment probe is visible in the fused image and the lesion is located at an angle from the treatment probe.

Clause 16. The method of any of clauses 11 to 15, wherein the lesion is shown at the radial distance in a fused longitudinal image.

Clause 17. The method of any of clauses 11 to 16, wherein the lesion is projected onto the fused image at a distance from a treatment probe that is greater than a vector projection of the lesion onto the fused image.

Clause 18. The method of any of clauses 11 to 17, wherein the fused image comprises a fused longitudinal image and wherein the lesion is projected onto the fused longitudinal image and a boundary of a three dimensional treatment profile is overlaid on the fused longitudinal image with a plurality of user inputs and markers configured for a user to adjust the three dimensional treatment profile shown on the fused longitudinal image.

Clause 19. The method of any of clauses 11 to 18, wherein the fused image comprises a fused transverse image and wherein the lesion is projected onto the fused transverse image and a boundary of a three dimensional treatment profile is overlaid on the fused transverse image with a plurality of user inputs and markers configured for a user to adjust the three dimensional treatment profile shown on the fused transverse image.

Clause 20. The method of any of clauses 11 to 19, wherein the fused image comprises a fused longitudinal image and a fused transverse image and wherein the lesion is projected onto the fused longitudinal image and the fused transverse image, and a boundary of a three dimensional treatment profile is overlaid on the fused longitudinal image and the fused transverse image with a plurality of user inputs and markers configured for a user to adjust the three dimensional treatment profile.

Clause 21. The method of any of clauses 11 to 20, wherein the treatment probe is visible in the intraoperative image and not in the diagnostic image

Clause 22. The method of any of clauses 11 to 21, wherein image data from the diagnostic image is fused with data from the intraoperative image to generate fused image data.

Clause 23. The method of any of clauses 11 to 22, wherein the pre-operative image data is fused with the intraoperative image data.

Clause 24. The method of any of clauses 11 to 23, wherein an elongate imaging probe is translated along a longitudinal axis to a plurality of locations along the axis to generate a plurality of transverse intraoperative images and wherein the elongate imaging probe is rotated around the longitudinal axis to generate a plurality of longitudinal intraoperative images.

Clause 25. The method of any of clauses 11 to 24, wherein a rotational angle of the elongate imaging probe about the elongate axis remains fixed among the plurality of transverse images and wherein a translational position of the elongate imaging probe remains fixed among the plurality of longitudinal images.

Clause 26. The method of any of clauses 11 to 25, wherein a model is generated with data from the diagnostic image and optionally wherein the model comprises a virtual object.

Clause 27. The method of any of clauses 11 to 26, wherein the diagnostic image is generated without a probe placed in the patient and the intraoperative image is generated with the probe placed in the patient.

Clause 28. The method of any of clauses 11 to 27, wherein diagnostic image data is projected onto the intraoperative image data in response to a shape of the probe placed in the patient.

Clause 29. The method of any of clauses 11 to 28, wherein the probe comprises a substantially stiff, straight probe inserted into a body lumen so as to straighten the body lumen and wherein the diagnostic image data is projected onto the intraoperative image data in response to the probe having straightened the body lumen.

Clause 30. The method of any of clauses 11 to 29, wherein the lumen comprises a urethra and the probe comprises a treatment probe.

Clause 31. The method of any of clauses 11 to 30, further comprising: acquiring a plurality of 2D intraoperative images to generate a 3D intraoperative image; fusing 3D diagnostic image data with the 3D intraoperative image to generate a 3D fused image; generating a 3D treatment plan in response to the 3D fused image; providing a real time 2D image on a display for a user to monitor treatment.

Clause 32. The method of any of clauses 11 to 31, wherein the real time 2D image comprise a real time fused 2D image and optionally wherein the real time fused 2D image comprises a real sagittal image with the lesion projected thereon with a plurality of markers.

Clause 33. The method of any of clauses 11 to 32, wherein the 3D treatment plan is presented to the user with a plurality of markers shown on a plurality of transverse images from the fused 3D image and the user interface is configured for the user to adjust the plurality of markers and the treatment profile shown on each of the plurality of fused transverse images.

Clause 34. The method of any of clauses 11 to 33, wherein the steps of acquiring, fusing, generating and providing are performed in sequence.

Clause 35. A method of generating a three dimensional (3D) treatment plan, the method comprising: receiving 3D diagnostic image data; generating a 3D intraoperative image; fusing 3D diagnostic image data with 3D intraoperative image data to generate a fused 3D image; generating a plurality of fused two dimensional (2D) images from the fused 3D image; generating a 2D treatment plan in response to data from each of the plurality of fused 2D images; and generating a 3D treatment plan in response to the plurality of 2D treatment plans.

Clause 36. The method of any of clauses 11 to 35, wherein each of the plurality of fused 2D images is presented to the user on a display with a plurality of markers overlaid thereon and the user interface is configured for the user to adjust the plurality of markers and the treatment profile displayed on said each of the plurality of fused images.

Clause 37. The method of any of clauses 11 to 36, wherein the plurality of fused 2D images comprise Digitally Reconstructed Radiographs generated from the fused 3D image.

Clause 38. The method of any of clauses 11 to 37, wherein the plurality of fused images comprises a plurality of fused transverse images generated from the fused 3D image.

Clause 39. The method of any of clauses 11 to 38, further comprising providing a real time 2D image on a display for a user to monitor treatment.

Clause 40. The method of any of clauses 11 to 39, wherein the real time 2D image comprise a real time fused 2D image and optionally wherein the real time fused 2D image comprises a real time sagittal image with the lesion projected thereon with a plurality of markers.

Clause 41. The method of any of clauses 11 to 40, wherein the plurality of fused 2D images is processed with an AI algorithm to generate the 2D treatment plan on said each of the plurality of fused 2D images.

Clause 42. The method of any of clauses 11 to 41, wherein the user interface is configured to display the AI generated 2D treatment plan on said each of the plurality of fused 2D images and configured to receive inputs for the user to adjust the AI generated 2D treatment plan and corresponding markers on said each of the plurality of fused 2D images.

Clause 43. The method of any of clauses 11 to 42, wherein the user interface is configured to display the 2D treatment plan on said each of the plurality of fused 2D images and configured to receive inputs for the user to adjust the 2D treatment plan and corresponding markers on said each of the plurality of fused 2D images.

Clause 44. A computer readable storage medium comprising instructions that, when executed by a processor, perform the method of any one of the preceding clauses.

Clause 45. An apparatus, the apparatus comprising: a display; a processor comprising the computer readable storage medium of clause 44.

Clause 46. The method of any of the preceding clauses, further comprising treating the patient with the energy source.

Clause 47. The method or apparatus of any one of the preceding clauses, wherein the intraoperative image comprises one or more of an ultrasound image, a three-dimensional (3D) ultrasound image, a longitudinal ultrasound image, a sagittal ultrasound image, a transverse ultrasound image, a trans rectal ultrasound (TRUS) image, a longitudinal TRUS image, a sagittal TRUS image or a transverse TRUS image.

Clause 48. The method or apparatus of any of the preceding clauses, wherein a user provides an input by touching a display and a treatment marker shown on the display moves in response to the user input.

Clause 49. The method or apparatus of any one of the preceding clauses, wherein an image plane is located away from a corresponding location of a lesion and the lesion is projected onto the image plane in the fused image and optionally wherein the projected lesion comprises a ghost image.

Clause 50. The method or apparatus of any one of the preceding clauses, wherein a plane of an intraoperative image is located away from a corresponding location of a lesion present in the diagnostic image and the lesion projected onto the fused image comprises a ghost lesion and optionally because the ghost lesions is located away from the plane of the intraoperative image.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A method of generating a treatment plan, the method comprising:
receiving a diagnostic image of a patient, the diagnostic image comprising one or more lesions;
receiving an intraoperative image of the patient; and
combining data from the diagnostic image with data from the intraoperative image to generate a fused image with the lesion projected onto the fused image.

2. The method of claim 1, further comprising:
determining a location of a treatment probe and projecting the lesion onto the fused image in response to the location of the treatment probe.

3. The method of claim 1, wherein the lesion is located a radial distance from the treatment probe and the lesion is projected onto the fused image at the radial distance from the treatment probe to the lesion.

4. The method of claim 3, wherein the intraoperative image extends along a plane and the lesion is located away from the plane.

5. The method of claim 4, wherein the treatment probe is visible in the fused image and the lesion is located at an angle from the treatment probe.

6. The method of claim 3, wherein the lesion is shown at the radial distance in a fused longitudinal image.

7. The method of claim 1, wherein the lesion is projected onto the fused image at a distance from a treatment probe that is greater than a vector projection of the lesion onto the fused image.

8. The method of claim 1, wherein the fused image comprises a fused longitudinal image and wherein the lesion is projected onto the fused longitudinal image and a boundary of a three dimensional treatment profile is overlaid on the fused longitudinal image with a plurality of user inputs and markers configured for a user to adjust the three dimensional treatment profile shown on the fused longitudinal image.

9. The method of claim 1, wherein the fused image comprises a fused transverse image and wherein the lesion is projected onto the fused transverse image and a boundary of a three dimensional treatment profile is overlaid on the fused transverse image with a plurality of user inputs and markers configured for a user to adjust the three dimensional treatment profile shown on the fused transverse image.

10. The method of claim 1, wherein the fused image comprises a fused longitudinal image and a fused transverse image and wherein the lesion is projected onto the fused longitudinal image and the fused transverse image, and a boundary of a three dimensional treatment profile is overlaid on the fused longitudinal image and the fused transverse image with a plurality of user inputs and markers configured for a user to adjust the three dimensional treatment profile.

11. The method of claim 1, wherein the treatment probe is visible in the intraoperative image and not in the diagnostic image.

12. The method of claim 1, wherein image data from the diagnostic image is fused with data from the intraoperative image to generate fused image data.

13. The method of claim 1, wherein the pre-operative image data is fused with the intraoperative image data.

14. The method of claim 1, wherein the diagnostic image is generated without a probe placed in the patient and the intraoperative image is generated with the probe placed in the patient.

15. The method of claim 14, wherein diagnostic image data is projected onto the intraoperative image data in response to a shape of the probe placed in the patient.

16. The method of claim 15, wherein the probe comprises a substantially stiff, straight probe inserted into a body lumen so as to straighten the body lumen and wherein the diagnostic image data is projected onto the intraoperative image data in response to the probe having straightened the body lumen.

17. The method of claim 1, further comprising:
acquiring a plurality of 2D intraoperative images to generate a 3D intraoperative image;
fusing 3D diagnostic image data with the 3D intraoperative image to generate a 3D fused image;
generating a 3D treatment plan in response to the 3D fused image;
providing a real time 2D image on a display for a user to monitor treatment.

18. The method of claim 17, wherein the real time 2D image comprise a real time fused 2D image and optionally wherein the real time fused 2D image comprises a real sagittal image with the lesion projected thereon with a plurality of markers.

19. The method of claim 17, wherein the 3D treatment plan is presented to the user with a plurality of markers shown on a plurality of transverse images from the fused 3D image and the user interface is configured for the user to adjust the plurality of markers and the treatment profile shown on each of the plurality of fused transverse images.

20. The method of claim 17, wherein the steps of acquiring, fusing, generating and providing are performed in sequence.
